(19) United States
(12) Patent Application Publication
(10) Pub. No.: US 2022/0192529 A1
(43) Pub. Date: Jun. 23, 2022

(54) PULSE RECOGNITION AND BLOOD OXYGEN SATURATION SYSTEMS AND METHODS

(71) Applicant: Masimo Corporation, Irvine, CA (US)

(72) Inventors: Ammar Al-Ali, San Juan Capistrano, CA (US); Sung Uk Lee, Irvine, CA (US); Anmol B. Majmudar, Irvine, CA (US); Walter M. Weber, Laguna Hills, CA (US)

(21) Appl. No.: 17/450,822

(22) Filed: Oct. 13, 2021

Related U.S. Application Data
(60) Provisional application No. 63/134,096, filed on Jan. 5, 2021.
(60) Provisional application No. 63/134,076, filed on Jan. 5, 2021.
(60) Provisional application No. 63/133,632, filed on Jan. 4, 2021.
(60) Provisional application No. 63/091,817, filed on Oct. 14, 2020.
(60) Provisional application No. 63/091,476, filed on Oct. 14, 2020.
(60) Provisional application No. 63/091,494, filed on Oct. 14, 2020.
(60) Provisional application No. 63/091,605, filed on Oct. 14, 2020.

(51) Int. Cl. A61B 5/024; A61B 5/00; A61B 5/1455; A61B 5/11

(52) U.S. Cl. CPC A61B 5/02433; A61B 5/0004; A61B 5/0017; A61B 5/681; A61B 2562/0219; A61B 5/7225; A61B 5/14551; A61B 5/11; A61B 5/7203

(57) ABSTRACT

A system for determining blood oxygen saturation of a user from a signal responsive to light absorption by tissue of a monitored patient and determine physiological parameters based on the signal. In some examples, the signal may be analyzed in conjunction with motion data, which may include gyroscope and accelerometer data, to determine a pulse rate and/or blood oxygen saturation value.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

This disclosure relates to pulse oximetry, including wrist-based pulse oximetry and pulse oximetry in the presence of motion.

### BACKGROUND

Pulse oximeters are inherently vulnerable to physical movement by the wearer. The movement introduces noise that can obscure the subtle signals used to determine an oxygen saturation measurement. Masimo Corporation of Irvine Calif. developed the first measure through motion system marketed under the name Masimo Signal Extraction Technology or SET. SET was designed measure through the types of motions typically found in a hospital setting. SET was also designed to be used with sensors worn on body parts with a high density of capillary beds (finger, ear, toe, nose, etc.).

### SUMMARY

While measure through motion processing is useful in certain parts of the body with a high density of capillary beds, the wrist presents challenges to current measure through motion technology due to its low capillary density and weak signal. Current measure through motion processing was also not designed for normal everyday movements, such as exercising or significant movements not normally occurring in a hospital setting.

In some examples, a system for determining a plurality of physiological parameters of a user from a signal responsive to light absorption by tissue of a monitored patient can include an electronic signal processor configured to: receive data from a plurality of sensors comprising at least one noninvasive optical sensor and at least one motion sensor, wherein said received data is responsive to light attenuated by tissue of the user; and determine a plurality of physiological parameters using a plurality of parameter engines configured to calculate one or more physiological parameters based on the received data.

In some examples, a system for determining pulse rate of a user from a signal responsive to light absorption by tissue of a monitored patient can include an electronic signal processor configured to: receive one or more optical signals comprising a red PPG signal, an IR PPG signal, and a green PPG signal; receive one or more motion signals comprising at least one gyroscope signal and at least one accelerometer signal; determine at least one first candidate pulse rate from the green PPG signal using the one or more motion signals; if a minimum peak of the one or more motion signals is greater than a first minimum frequency: apply a first bandpass filter to the green PPG signal to select signal above the first minimum frequency; and determine at least one second candidate pulse rate from the first filtered green PPG signals using the one or more motion signals as a noise reference; if a minimum peak of the one or more motion signals is greater than a second minimum frequency: apply a second bandpass filter to the green PPG signal to select signal above the second minimum frequency; and determine at least one third candidate pulse rate from the second filtered green PPG signal using the green PPG signal as a self-reference; determine at least one fourth candidate pulse rate from the green PPG signal using a red signal as a noise reference; determine at least one fifth candidate pulse rate from the green PPG signal using an infrared signal as a noise reference; and select a final pulse rate from among the at least one first, second, third, fourth, and fifth candidate pulse rates.

In some examples, a system for determining pulse rate of a user from a signal responsive to light absorption by tissue of a monitored patient can include an electronic signal processor configured to: receive one or more optical signals comprising a red PPG signal, an IR PPG signal, and a green PPG signal; receive one or more motion signals comprising at least one gyroscope signal and at least one accelerometer signal; determine a plurality of pulse rate candidates using a plurality of pulse rate engines; select a first candidate pulse rate associated with a low motion engine if motion falls below a threshold level; perform one or more time domain feature checks to determine the accuracy of the first candidate pulse rate; determine a type and elapsed time of a motion present in the one or more optical signals or one or more motion signals; determine a set of boundaries for a pulse rate based on the type and elapsed type of the motion; select a second candidate pulse rate associated with a motion engine if the pulse rate falls within the set of boundaries; and output the selected first or second candidate pulse rates as a final pulse rate.

In some examples, a system for determining an SpO2 value of a user from a signal responsive to light absorption by tissue of a monitored patient can include an electronic signal processor configured to: receive one or more optical signals comprising a red PPG signal, an IR PPG signal, and a green PPG signal; receive one or more motion signals comprising at least one gyroscope signal and at least one accelerometer signal; determine a plurality of SpO2 candidate values using a plurality of saturation engines; select a first candidate SpO2 value from the plurality of SpO2 candidate values; determine a baseline saturation tracker based on a DC component of a red PPG signal and a DC component of an infrared PPG signal; determine if there is excessive motion interference; output the first candidate SpO2 value if there is no excessive motion interference; select a second candidate SpO2 value based on one or more features in the received data and the baseline saturation tracker to output; update the baseline saturation tracker; and output the selected first or second candidate SpO2 values as a final SpO2 value.

In some examples, a system for determining an SpO2 value of a user from a signal responsive to light absorption by tissue of a monitored patient can include an electronic signal processor configured to: receive one or more optical signals comprising a red PPG signal, an IR PPG signal, and a green PPG signal; receive one or more motion signals comprising at least one gyroscope signal and at least one accelerometer signal; project red PPG and infrared PPG signals respectively onto green PPG signal subspace; and subtract the projection from the red PPG signal and the infrared PPG signal to determine at least one noise reference; use a noise canceller to remove green PPG signal from the red PPG signal and infrared PPG signal to determine at least one noise reference; use a noise canceller to remove the at least one noise reference from the red PPG signal and infrared PPG signal to generate a clean red PPG signal and clean infrared PPG signal; and determine a candidate SpO2 value based on a ratio of the clean infrared PPG signal and clean red PPG signal.

In some examples, a system for determining an SpO2 value of a user from a signal responsive to light absorption by tissue of a monitored patient, said system comprising an electronic signal processor configured to: receive one or more optical signals comprising a red PPG signal, an IR PPG signal, and a green PPG signal; receive one or more motion signals comprising at least one gyroscope signal and at least one accelerometer signal; project red PPG or infrared PPG signals onto green PPG signal subspace; and subtract the projection from the red PPG or infrared PPG signal to determine at least one noise reference; use a noise canceller to remove the at least one noise reference from the red PPG signal and infrared PPG signal to generate a clean red PPG signal and clean infrared PPG signal; and determine a candidate SpO2 value based on a ratio of the clean infrared PPG signal and clean red PPG signal.

### BRIEF DESCRIPTION OF THE DRAWINGS

The drawings and the associated descriptions are provided to illustrate embodiments of the disclosure and not to limit the scope of the claims.

FIG. 1 illustrates an example wearable device including a plethysmograph module worn on a wrist.

FIG. 2A is a schematic system diagram illustrating a wearable device including a plethysmograph module.

FIG. 2B illustrates an aspect of the subject matter in accordance with one embodiment.

FIG. 3A illustrates a bottom perspective view of an example plethysmograph module of a wearable device.

FIG. 3B illustrates a bottom view of the example plethysmograph module of FIG. 3A.

FIG. 3C illustrates a side view of the example plethysmograph module of FIG. 3A.

FIG. 4A illustrates a top view of an example embodiment of a physiological parameter measurement sensor or module.

FIG. 4B illustrates an example physiological parameter measurement sensor or module and example light paths between emitters and detectors of the module.

FIG. 4C illustrates a longitudinal cross-sectional view of an example physiological parameter measurement sensor or module.

FIG. 4D illustrates a longitudinal cross-sectional, that is orthogonal to the view of FIG. 4C, of an example physiological parameter measurement sensor or module.

FIG. 5 to FIG. 6 illustrate example displays of physiological parameter(s) measured by the wearable device.

FIG. 7 shows a block diagram of an example sensor device, such as a wearable device.

FIG. 8 illustrates an example controller that may be configured to processes one or more input values to produce the one or more outputs, such as a blood oxygen saturation and a pulse rate.

FIG. 9 illustrates an example of how one or more engines of a controller may interact to produce one or more outputs.

FIG. 10 illustrates an example pulse rate determination engine.

FIG. 11 illustrates an example of corresponding green, IR, and Red PPG signals.

FIG. 12 illustrates an example noise reduction engine that may be part of a pulse rate determination engine.

FIG. 13, FIG. 14, and FIG. 15 illustrates example green PPG signals, accelerometer signals, and gyroscope signals that may be received as input.

FIG. 16 illustrates example decision logic that may be used to select a candidate pulse rate from a plurality of sub-engines that are configured to produce a two candidate pulse rates and corresponding confidence scores.

FIG. 17A and FIG. 17B illustrate example thresholds that may be used as part of a decision logic for a noise reduction engine.

FIG. 18 and FIG. 19 illustrate various aspects of decision logic that may be used to select a candidate pulse rate from one or more of the candidate pulse rates determined by the one or more pulse rate engines.

FIG. 20 illustrates example empirical data across a range of users over illustrates a change in heart rate for a running motion type.

FIG. 21 illustrates an example SpO2 determination engine.

FIG. 22A and FIG. 22B illustrate example processes that may be used by a noise reference saturation engine to determine a signal reference saturation estimation and/or a noise/signal reference saturation estimation.

FIG. 23 illustrates example empirical data in the frequency domain that may be analyzed by a frequency domain saturation engine to determine a saturation estimate.

FIG. 24 and FIG. 25 illustrate various aspects of decision logic that may be used to select a candidate saturation value from one or more of the candidate saturation values determined by the one or more saturation engines.

FIG. 26 illustrates an example motion processing algorithm that may be part of a pre-processing engine.

FIG. 27 and FIG. 28 illustrate example gyroscope and accelerometer signals without and with processing to generate signals at a desired sample rate.

FIG. 29 illustrates an example error reduction process.

### DETAILED DESCRIPTION

Although certain embodiments and examples are described below, those of skill in the art will appreciate that the disclosure extends beyond the specifically disclosed embodiments and/or uses and obvious modifications and equivalents thereof based on the disclosure herein. Thus, it is intended that the scope of the disclosure herein disclosed should not be limited by any particular embodiments described below.

In finger-based pulse oximetry, light from sensors scatters around the bone and fatty tissue through rich capillary beds. The scattered light has a relatively constant path length, which is important in a ratio approach to pulse oximetry. In measuring physiological parameters from a wrist, however, light may be flooded over a larger surface area having few capillaries and scattered light may not have a constant path length. Additionally, while a finger has a lot of capillaries and no significant arteries or veins, a wrist has few capillaries and includes arteries and veins. Veins in particular are problematic due to their relative proximity to the surface and much higher attenuation resulting in increased physiologic noise. There is a certain amount of positional randomness in light scattering from a vein or artery. Additionally, perfusion on a wrist may be significantly lower than perfusion on a finger, making pulse oximetry much more difficult on a wrist. Thus, a different approach to pulse oximetry on a wrist over a finger is needed in order to account for these differences. Described herein are systems and methods for pulse oximetry on an area of the body other than a finger or digit, such as a wrist. Advantageously, systems and methods described herein may allow for more accurate and consistent measurement of physiological parameters using a non-invasive sensor in a smart watch or other device configured to measure parameters from the body of a user.

### Overview of Wearable Device Including a Plethysmograph Module

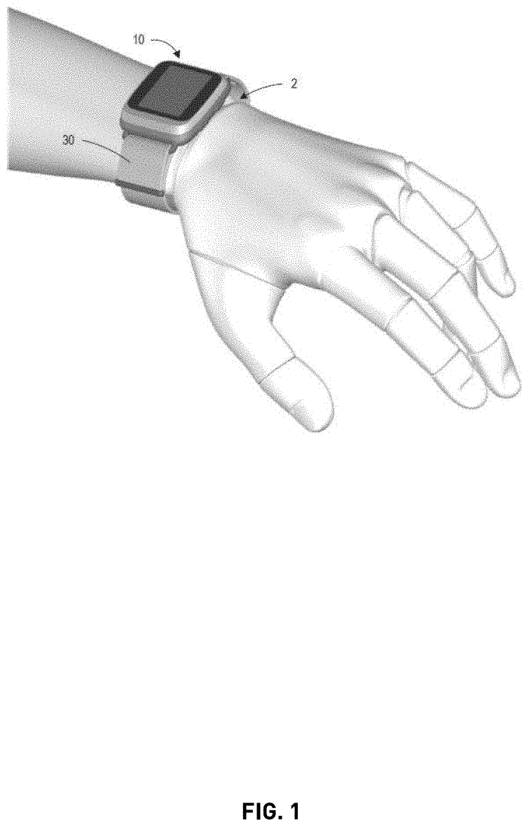

Daily use of a wearable plethysmography monitoring device, which can monitor oximetry-based physiological parameters, can be beneficial to the wearer. The device, such as a device 10 as shown in FIG. 1, can be a wristwatch (also referred to as a “watch”) incorporating a plethysmograph sensor or a wrist-worn plethysmograph sensor with built-in watch or time-indicating functions. The device 10 can include an adjustable strap 30 to releasably secure the device 10 around the wrist 2 of the wearer. The wearer can be informed of physiological parameters, such as vital signs including but not limited to heart rate and oxygen saturation (SpO2). Of course the present specification is not limited solely to a watch, but can include other embodiments. For example, the sensor can be worn on the wrist without a watch, screen or other smartwatch features.

### Various Example Components of the Wearable Device

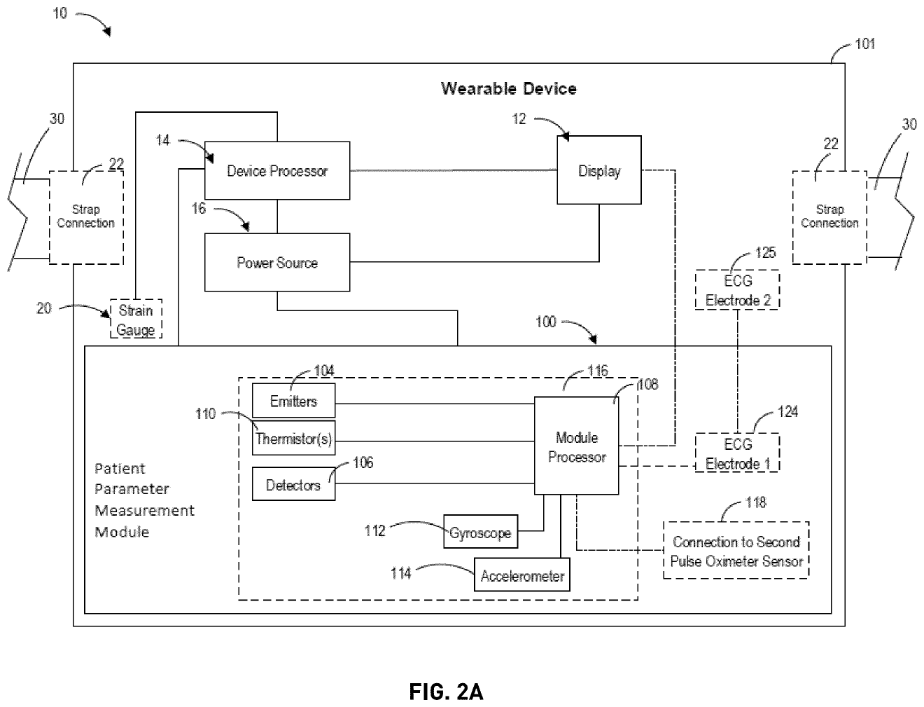

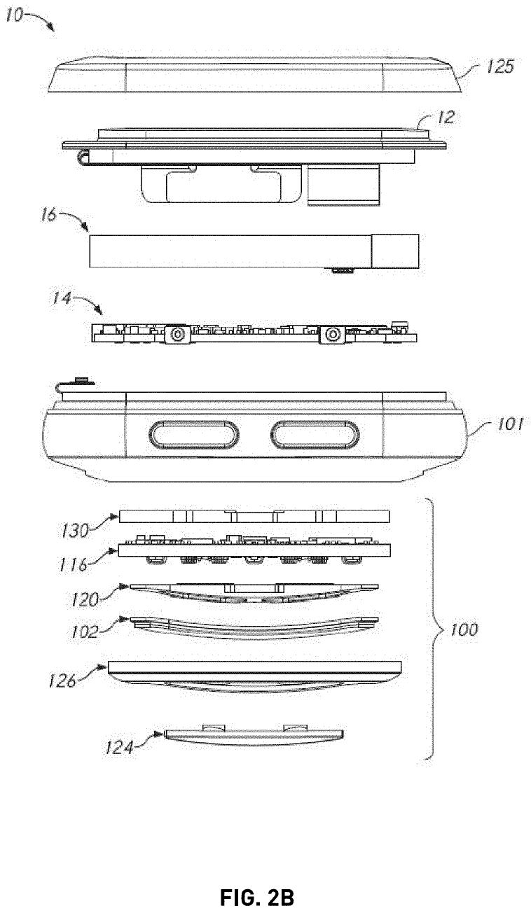

Components of the wearable device will now be described. As shown in FIG. 2A and FIG. 2B, the device 10 can include its own device processor 14, which can be a digital/analog chip or other processor(s), such as a digital watch processor or a smartwatch processor. As shown in FIG. 2B, the device processor 14 can be located on a PCB. As shown in FIG. 2A and FIG. 2B, the device 10 can include a power source 16, which can be a battery, for powering the device processor 14, the display screen 12, and/or the physiological parameter measurement module 100. The battery 16 can last at least 10 hours, or at least 12 hours, or at least 14 hours, or at least about 16 hours after each charge, with continuous measurements and/or displaying of certain physiological parameters, such as SpO2 and pulse rate.

The device 10 can be configured to display time after the battery 16 has been depleted, even if other features (for example, measuring physiological parameters using the module) may not be available when the battery 16 has been depleted. Additionally, when the device 10 is used clinically, the display 12 can also continue displaying critical patient information (for example, the patient's name, date of admission, etc.) after the battery 16 has been depleted. The device 10 may include nonvolatile memory to store the critical patient information. The device 10 can include a dual-battery configuration with a main battery and a backup battery. Power management of the device 10 may switch automatically for the device 10 to be powered by the backup battery when the main battery has been depleted. The device can additionally or alternatively be configured to be solar-powered, for example, by including a solar panel on the dial or elsewhere of the wearable device 10. The display 12 of the device 10 can use e-ink or ULP (ultra low power screen) technology, which draws little amount of current for displaying information. The display 12 may automatically adjust the brightness, being brighter when outdoors and dimmer when indoors to further prolong battery life.

As shown in FIG. 2A and FIG. 2B, the sensor or module 100 of the wearable device 10 can include a sensor or module processor 108 (which can include a memory and/or other electronics). The sensor or module processor 108 can process signals from one or more of the sensors in the sensor or module 100 (or optionally other sensors in communication with the device 10) to determine a plurality of physiological parameters. All the processing of the raw sensor data of the sensors in communication (via a wired and/or wireless connection) with the sensor or module processor 108 may be performed by the sensor or module processor 108. The sensor or module processor 108 can be configured to drive the emitters 104 to emit light of different wavelengths and/or to process signals of attenuated light after absorption by the body tissue of the wearer from the detectors 106. The sensor or module processor 108 can determine and output for display on the device display screen 12 the physiological parameters based on the detected signals. Optionally, the sensor or module 100 can send the signals from the detectors 106 (for example, preprocessed signals) to the device processor 14, which can determine and output for display the physiological parameters based on the detected signals. The absorption of light can be via transreflectance by the wearer's body tissue, for example, by the pulsatile arterial blood flowing through the capillaries (and optionally also the arteries) within a tissue site where the device 10 is worn (for example, the wrist). The sensor or module processor 108 can be located on a PCB 116, such as shown in FIG. 2B.

The module 100 configured to measure an indication of the wearer's physiological parameters. This can include, for example, pulse rate, respiration rate, SpO2, Pleth Variability Index (PVI), Perfusion Index (PI), Respiration from the pleth (RRp), total hemoglobin (SpHb), hydration, glucose, blood pressure, and/or other parameters. The module 100 can perform intermittent and/or continuous monitoring of the measured parameters. The module 100 can additionally and/or alternatively perform a spot check of the measured parameters, for example, upon request by the wearer.

The sensor or module 100 can include more than one group or cluster of light emitters (such as LEDs) 104 and more than one group of photodetectors (also referred to as “detectors”) 106. Each group of emitters 104 can be configured to emit four (or more) different wavelengths described herein. The sensor or module 100 can include one or more thermistors 110 or other types of temperature sensors. The thermistor(s) 110 can be placed near one or more groups of emitters 104. There can be at least one thermistor 110 near each group of emitters 104. The thermistor(s) 110 can provide for wavelength correction of the light emitted by the emitters 104. Optionally, the thermistor(s) 110 can additionally measure a temperature of the wearer of the device 10. Optionally there can be one or more thermistors 110 located at other places of the sensor or module 100. The emitters 104, the thermistor(s) 110, and/or the detectors 106 can be positioned on the PCB 116.

The emitters 104 of the plethysmograph module 100 can be configured to emit a plurality of (for example, three, four, or more) wavelengths. The emitters 104 can be configured to emit light of a first wavelength providing an intensity signal that can act as a reference signal. The first wavelength can be more absorbent by the human body than light of other wavelengths emitted by the emitters 104. The reference signal can be used by the plethysmograph module processor 108 to extract information from the other signals, for example, information relevant to and/or indicative of the pulsing rate, harmonics, or otherwise. The plethysmograph module processor 108 can focus the analysis on the extracted information for calculating the physiological parameters of the wearer. The first wavelength can include a range of wavelengths, including, for example, from about 530 nm to about 650 nm, or from about 580 nm to about 585 nm, or from about 645 nm to about 650 nm, or about 580 nm, or about 645 nm. The light providing the reference signal can have an orange color. Alternatively, the light providing the reference signal can have a green color.

The emitters 104 can be configured to emit light of a second wavelength having a red or orange color. The second wavelength can be from about 620 nm to about 660 nm. Light of the second wavelength can be more sensitive to changes in SpO2. The second wavelength is preferably closer to 620 nm, which results in greater absorption by the body tissue of the wearer, and therefore a stronger signal and/or a steeper curve in the signal, than a wavelength that is closer to 660 nm. The plethysmograph module processor 108 can extract information such as the pleth waveform from signals of the second wavelength. The emitters 104 can be configured to emit light of a third wavelength of about 900 nm to about 970 nm, or about 905 nm, or about 907 nm. The pulse oximeter processor can use the third wavelength as a normalizing wavelength when calculating ratios of the intensity signals of the other wavelengths.

Additionally or optionally, the emitters 104 can be configured to emit light having a fourth wavelength that is more sensitive to changes in water than the rest of the emitted wavelengths. The fourth wavelength can be about 970 nm or higher than 970 nm. The plethysmograph module processor 108 can determine physiological parameters such as a hydration status of the wearer based at least in part on a comparison of the intensity signals of the fourth wavelength and a different wavelength detected by certain detectors 106.

The device 10 can optionally include one or more thermistors 110 or other types of temperature sensors. The thermistor(s) 110 can be placed near one or more groups of emitters 104. The thermistor(s) 110 can provide for wavelength correction of the light emitted by the emitters 104. Optionally, the thermistor(s) 110 can additionally measure a temperature of the wearer of the device 10. Optionally, there can be one or more thermistors 110 located at other places of the module 100. The emitters 104, the thermistor(s) 110, and/or the detectors 106 can be positioned on a printed circuit board (PCB) 116. The device 100 can include a gyroscope 112, an accelerometer 114, and/or other position and/or posture detection sensor(s). Optionally, the module processor 108, the gyroscope 112, and/or the accelerometer 114 can be located on the PCB 116. The gyroscope 112 and/or the accelerometer 114 can be in electrical communication with the module processor 108. Optionally, the device can include an electrocardiogram (ECG) sensor including a plurality of electrodes 124, 125 configured to make contact with the wearer's skin. One or more ECG electrodes 124 may be located on the module 100. One or more ECG electrodes 125 may be located elsewhere on the device 10. The ECG sensor can be in electrical communication with the module processor 108. The module processor 108 can process signals from one or more of the sensors in the module 100 to determine a plurality of physiological parameters. Optionally, all the processing of the raw sensor data of the sensors in communication with the module processor 108 is performed by the module processor 108.

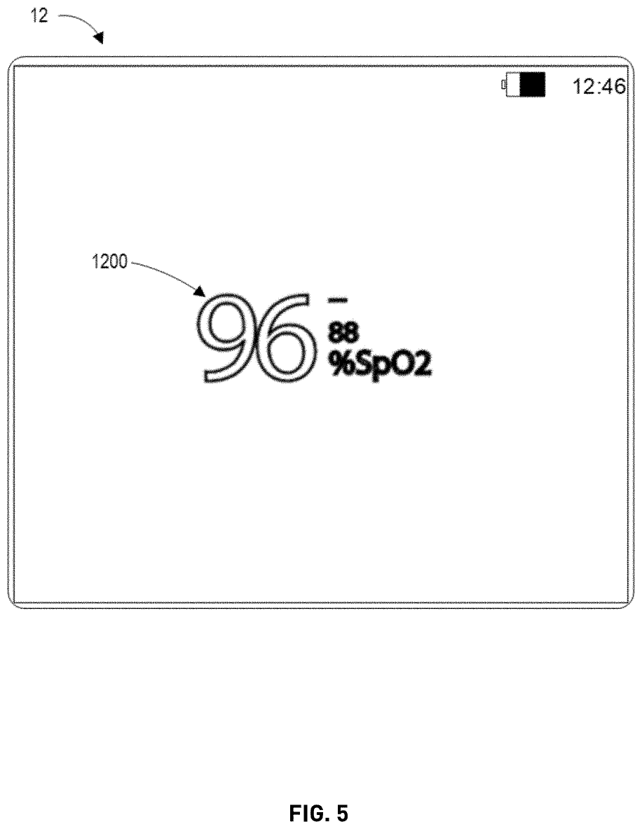

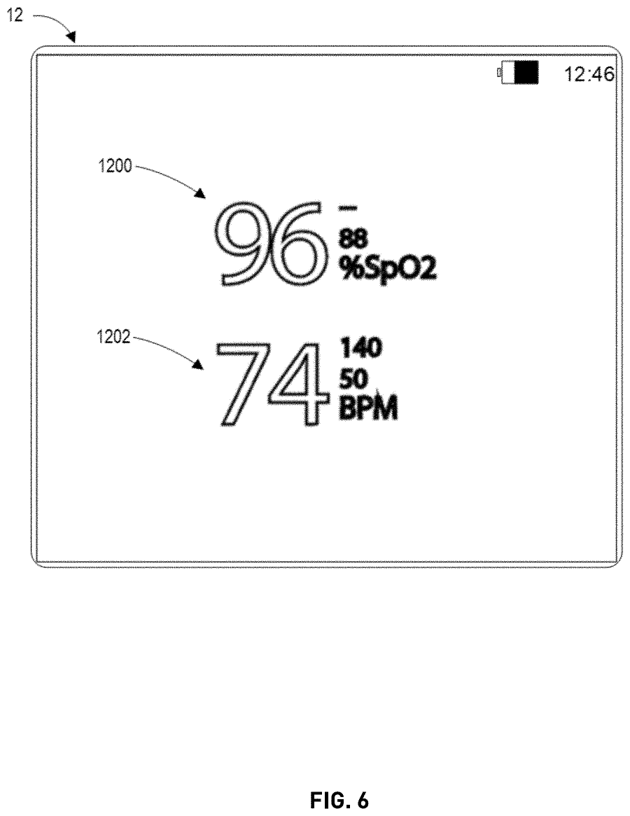

As shown in FIG. 2A, the device 10 can include its own device processor 14, which can be a digital/analog chip or other processor(s), such as a digital watch processor or a smartwatch processor. The device 10 can include a power source 16, which can be a battery, for powering the device processor 14, the display screen 12, and/or the plethysmograph module 100. FIG. 5 illustrates an example display of the wearer's SpO2 measurement 1200 on the display screen 12. FIG. 6 illustrates an example display of the wearer's SpO2 measurement 1200 and pulse rate measurement 1202 on the display screen 12. The display screen 12 can additionally or alternatively display other parameters, or combinations thereof, monitored by the module 100.

Optionally, the plethysmograph module 100 can be preassembled before being integrated into the device 10. An electrical connection can be established between the plethysmograph module PCB 116 and the circuit of the rest of the device 10, including for example, the device processor 14, the display 12, and the power source 16. The plethysmograph module 100 can be characterized before being assembled with the rest of the device 10. Alternatively, a housing of the plethysmograph module can be an integral component of a housing of the device.

As shown in FIG. 2A, the device 100 can include a gyroscope 112, an accelerometer 114, and/or other position and/or posture detection sensor(s). The gyroscope 112 and/or the accelerometer 114 can be in electrical communication with the sensor or module processor 108. The sensor or module processor 108 can determine motion information from signals from the gyroscope 112 and/or the accelerometer 114. The motion information can provide noise reference for analysis of the pleth information and other signal processing (for example, processing of ECG signals) performed by the sensor or module processor 108. The gyroscope 112 and/or the accelerometer 114 can be located on the PCB 116.

Optionally, as shown in FIG. 2A and FIG. 2B, the device 10 can include an electrocardiogram (ECG) sensor including a plurality of electrodes 124, 125 configured to make contact with the wearer's skin. One or more ECG electrodes 124 may be located on the sensor or module 100 (such as shown in FIG. 2B). One or more ECG electrodes 125 may be located elsewhere on the device (for example, an ECG electrode 125 can form a part of the housing of the wearable device 10 as shown in FIG. 2B). The ECG sensor can be in electrical communication with the sensor or module processor 108 via an ECG connector.

As shown in FIG. 2B, the physiological parameter measurement module 100 can include a skin-interfacing light transmissive cover 102 that encloses the first side of the PCB 116, which positions the plurality of light emitters 104 and detectors 106. The sensor or module 100 can include a light barrier construct 120 that is configured to divide the emitters 104 and the detectors 106 into different chambers such that light cannot travel or substantially cannot travel between the chambers. The light transmissive cover 102 can extend over the various emitter and detector chambers formed by the light barrier construct 120 and the PCB 116. The light transmissive cover 102 can include individual lenses or covers, a single lens or cover, or a combination of individual emitter chamber covering lenses or covers and a single lens or cover covering a plurality of detector chambers. In the example lens or cover 102b, the individual lenses or covers that are configured to cover the detector chambers can be interconnected with bridging portions 103 between the detector chambers, forming a single piece of lens or cover. The lens or cover 102b can be combined with the lenses or covers 102a covering the emitter chambers to cover all the openings in the light barrier construct 120 for forming sealed emitter and detector chambers. The light barrier construct 120 can be overmoulded to the lens or cover 102b and the lenses or covers 120a. The lens or cover 102b may not be configured to cover the emitter chambers, which can be covered by individual lenses, so as to avoid any light traveling between an emitter chamber and a detector chamber.

As shown in FIG. 2B, the physiological parameter measurement module 100 can include an opaque frame 126. The opaque frame 126 can accommodate the light barrier construct 120. Alternatively, the opaque frame 126 and the light barrier construct 120 can form an integral piece. The opaque frame 126 can include indentations having the shape and size to accommodate the ECG electrodes 124 or other components with a suitable shape and size. A front side of the electrodes 124 can have one or more posts 137 extending past openings in the opaque frame 126 into corresponding openings on the PCB 116. The posts 137 of the electrodes 124 can establish an electrical connection with the corresponding openings of the PCB 116. A plurality of screws (or other types of fasteners) can extend into the corresponding openings of the PCB 116 from the front side of the PCB 116 to secure the electrodes 124 to the sensor or module 100 by threadedly mating or otherwise with the posts 137. When a wearer puts the wearable device incorporating the sensor or module 100 onto the wearer's wrist, the electrodes 124 can make contact with the wearer's skin.

The tightness of the device 10 in the wearer's body (for example, the wrist) can be adjusted by adjusting any suitable strap(s) 30 used to secure the device 10 to the wearer's body. The strap(s) 30 can be connected to the device 10 using any suitable strap connections 22. For example, the strap connections 22 can be compatible with third party watch bands, wearable blood pressure monitors, and/or the like. The adjustment of the strap 30 around the wearer's wrist can reduce and/or eliminate a gap between a tissue-facing surface of the plethysmograph module 100 and the wearer's skin to improve accuracy in the measurements. The device 10 can include an optional strain gauge 20 to measure a pressure of the device 10 on the wearer. The strain gauge 20 can be located in a device housing 101 between the plethysmograph module 100 and other components of the device 10, for example, the power source 16, the device processor 14, or otherwise. When the device 10 is worn on the wearer, for example, on the wrist, the pressure exerted by the module 100 against the tissue can be transmitted to and measured by the strain gauge 20. Readings from the strain gauge 20 can be communicated to the device processor 14, which can process the readings and output an indication of the pressure asserted by the device 10 on the wearer to be displayed on the display 12. Optionally, the device 10 can output a warning that the device 10 is worn too tight when the device 10 has determined that the wearer's SpO2 readings are decreasing by a certain percentage, at a certain rate, and/or at a certain rate within a predetermined amount of time.

The plethysmograph modules disclosed herein can include an optional connector 118 for receiving a second sensor, which can be a second plethysmograph sensor such as a fingertip sensor configured to monitor opioid overdose, or any other suitable noninvasive sensor, such as an acoustic sensor, a blood pressure sensor, or otherwise. The connector 118 can be oriented such that the second sensor can extend from a housing of the device 10 with reduced or no impingement of the tissue at the device/tissue interface, resulting in less or no effect of the connector 118 or the second sensor on the blood flow through the device measurement site.

### Example Plethysmograph Modules

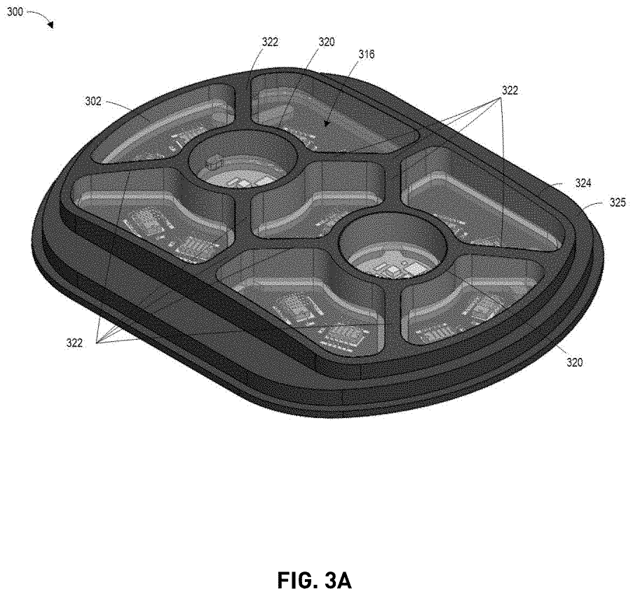

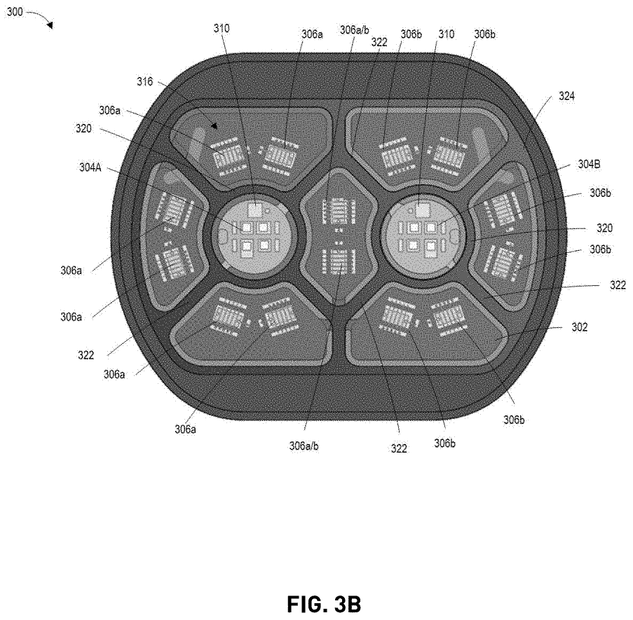

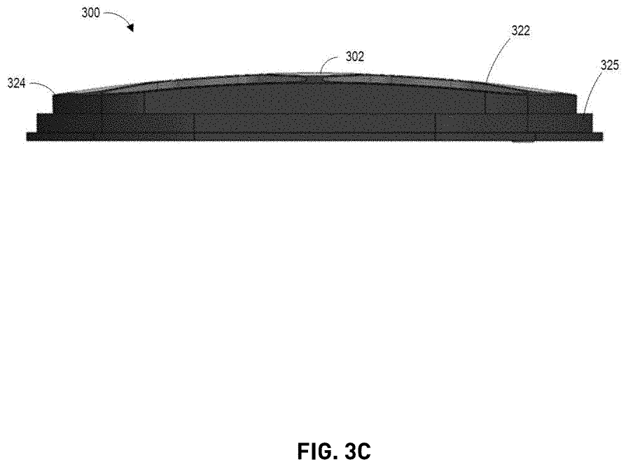

An example of the plethysmograph module described above can include two groups of emitters that are separated from each other by one or more light barriers, such as shown in FIG. 3A to FIG. 3B. The plethysmograph module 300 can include a first group of emitters 304a and a second group of emitters 304b. Each group of emitters can include four emitters (or optionally a different number of emitters, such as six or eight emitters). The emitters in each group 304a, 304b can emit at least the first, second, third, and fourth wavelengths as described above. The emitters 304a, 304b can be activated simultaneously and/or sequentially. The module 300 can include a temperature sensor 310 as described above near the first and second group of emitters 304a, 304b respectively.

The PCB 316 can be elliptical in shape. The two groups of the emitters 304a, 304b can be located on two parts of a generally central portion of the PCB 316 divided along the minor diameter of the ellipse. The shape of the PCB 316 can vary, for example, including oval, circular, rectangular, square, or otherwise. The first and second groups of emitters 304a, 304b can be located a distance from each other. The two groups of the emitters 304a, 304b can each be surrounded by a first light barrier 320.

The first and second groups of emitters 304a, 304b in the module 300 can be surrounded by two rings of detectors 306a, 306b that are separated from the first and second groups of emitters 304a, 304b by the first light barrier 320. The two rings of detectors 306a, 306b can share a plurality of (for example, two or more) detectors 306a/b common to both rings. The detectors 306a/b common to both rings can be located along the minor axis of the ellipse. In the illustrated example, the module 300 can include fourteen detectors, but the module 300 can also include a different total number of detectors.

The detectors 306a can be the far detectors for the second group of emitters 304b and the detectors 306a, 306a/b can be the near detectors for the first group of emitters 304a. The detectors 306b can be the far detectors for the first group of emitters 304a and the detectors 306b, 306a/b can be the near detectors for the second group of emitters 404b. Accordingly, each detector 306a, 306b, 306a/b can receive two signals for each wavelength emitted by the first and second groups of emitters 304a, 304b respectively. Signals outputted by the far and near detectors can provide different information due to the different light paths, which can travel through different areas of the tissue.

As shown in FIG. 3A and FIG. 3B, the detectors 306a, 306b, 306a/b can be separated or partitioned into seven detector regions by portions of the first light barrier 320 and second light barriers 322. Each detector region can include two detectors, or any other number of detectors. Along an outer perimeter of the module 300, the detectors 306a, 306b, 306a/b can be enclosed within a module side wall 324. A module processor of the module 300 can process signals from the detectors within the same detector region as one signal source. Accordingly, for each wavelength, the module processor can receive data from a total of fourteen signal sources, two from each detector region acting as the far and near detectors for the different groups of emitters respectively.

The plethysmograph module 300 can monitor a hydration status of the wearer using the DC bulk absorption measurements of the signals detected by the detectors. At the DC level, water can act as a light block for the fourth wavelength and as a lens for the other wavelengths. Each detector region can provide two measurements calculated from the signals received from the closer emitter group and the signals from the further emitter group. Signals detected at the far detectors can provide indication of the hydration status of the wearer. Signals detected at the near detectors can be used as reference or for comparison with the signals detected at the far detectors. The module processor can compare intensity signals of the fourth wavelength and another wavelength (for example, about 905 nm) that is less sensitive to changes in water from one of the far detectors. When the wearer's hydration status is in a normal range such that the wearer is not considered dehydrated in a medical sense, the signals of the fourth wavelength and the other wavelength can show the opposite trends, that is, one is increasing when the other one is decreasing. When the wearer becomes dehydrated in a medical sense, the opposite trends can become less distinct, for example, falling below a threshold.

As a person's hydration status is not expected to change rapidly, the plethysmograph module can optionally make a measurement of the hydration status less frequently than making measurements related to the wearer's pulse rate or SpO2 or other parameters. For example, the plethysmograph module processor can make a measurement of hydration status every 5 minutes, or longer, and/or upon (for example, only upon) a request by the wearer, such as when the wearer presses a button (physical and/or a touch button on the display 12) on the device or otherwise instructs the device using voice commands, hand gestures, and/or the like.

On the first side of the PCB 316, the module 300 can be enclosed by a curved lens or cover 302, which can include a single cover or multiple pieces of covers. As shown in FIG. 3C, the lens 302 can have a continuous curvature. The first and second light barriers 320, 322, and of the side wall 324 can extend from the first side of the PCB 316 to a tissue-facing surface of the module 300, generally following the curvature of the lens 302. The light barriers 320, 322 can extend to the lens 302 so that when the module 300 is pressed into the skin of the wearer of a device (see FIG. 1) incorporating the module 300, the upper surfaces of the first and second light barriers 320, 322, and of the side wall 324 can be configured to contact the skin of the wearer. The tissue-facing surface of the module 300 can include a continuous curvature. The pressure exerted by the curvature of the tissue-facing surface of the module 300 on the skin can increase a light illuminated and/or detection area, improve the optical coupling of the emitted light and the blood vessels and/or of the reflected light and the detectors, and/or reduce stagnation of the blood. The curvature can be configured so as to balance the pressure needed to improve contact between the tissue-facing surface of the module 300 and the skin, and the comfort of the wearer.

The PCB 316, the first and second light barriers 320, 322, the side wall 324, and the lens 302 can form a module housing. The first side of the PCB 316, which is enclosed by the module housing, can include the emitters 304a, 304b, detectors 306a, 306b, 306a/b, temperature sensor 310, and optionally any other sensors, for example, the gyroscope, the accelerometer, and/or the like. The second side of the PCB 316 opposite the first side can include the module processor, optionally any additional sensors, and other circuit hardware. On the second side of the PCB 316, the PCB 316 can be covered by melt plastic or other suitable electronics protective material.

The module housing can include a plurality of chambers separating the emitters groups and the detector regions such that light cannot travel between the chambers because of the various light barriers described herein. Light diffusing materials can be added above and around the emitters 304a, 304b, and/or optionally above and around the detectors 306a, 306b, 306a/b, to improve distribution of emitted lighted and/or detected light after attenuation by the tissue. The diffusing materials can be any suitable materials, for example, glass, ground glass, glass beads, opal glass, greyed glass, polytetrafluoroethylene, or a microlens-based, band-limited, engineered diffuser. The diffusing materials can include a flow of glass microsphere solution injected into the chambers and UV-cured. The lens 302 can also include glass microspheres. The light diffusing materials in the lens 302 and the chambers and/or the first light barrier 320 can make the emitted light leave the chambers enclosing the emitters 304a, 304b in a direction generally parallel to the height of the first light barrier 320. The light diffusing materials in the lens 302 and the other chambers can increase the amount of reflected light being directed to and detected by the detectors 306a, 306b, 306a/b.

### Example Plethysmograph Modules

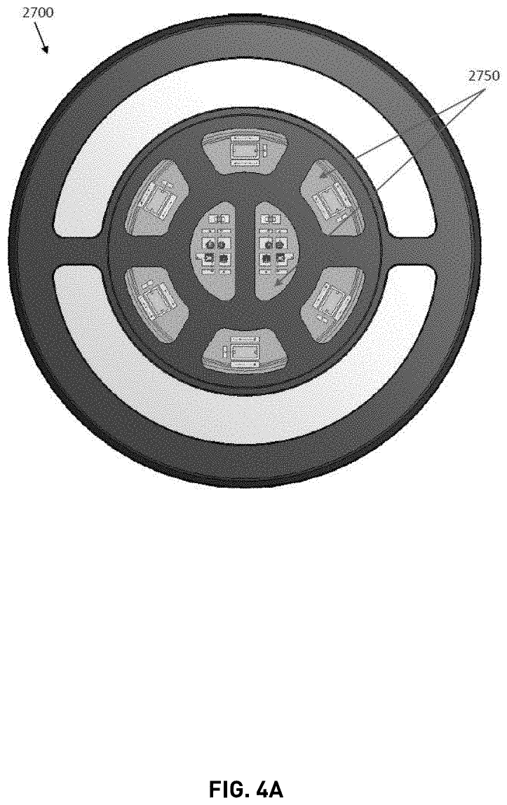

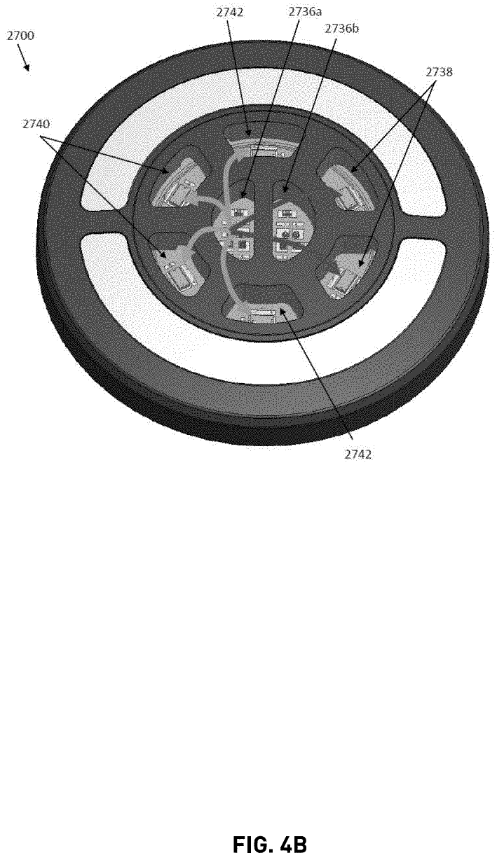

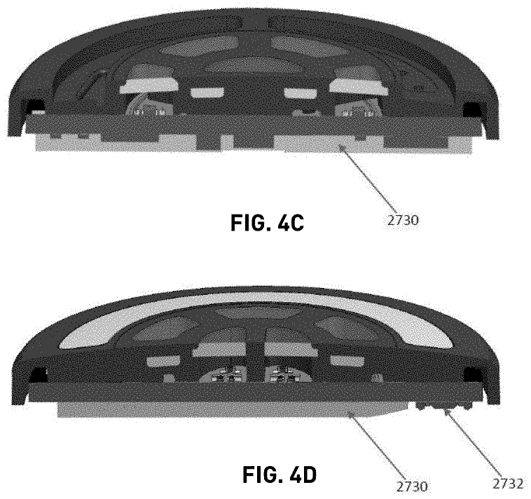

FIG. 4A illustrates an additional example configuration of a sensor or module. In some examples, the opaque frame 2726 may be configured such that the fill holes 2744 and channels 2746 allow the light diffusing materials to fill only the emitter chambers, or only the detector chambers, or both the emitter and detector chambers. As shown in FIG. 4A, in addition or alternative to the light diffusing materials, a bottom surface of each emitter and/or detector chambers can include a light-reflective surface material 2750. Optionally, as shown in FIG. 4A, substantially an entire central area of the PCB 2716 may include the light-reflective surface 2750. The light-reflective surface material 2750 can help in focusing the reflected light onto the detector inside each detector chamber to improve the amount of light captured by the detector. The light-reflective surface material 2750 can help in better distributing the light emitted by the emitters inside each emitter chamber to further facilitate making the light emitted by the different emitters in each emitter chamber appear as if coming from a single point source.

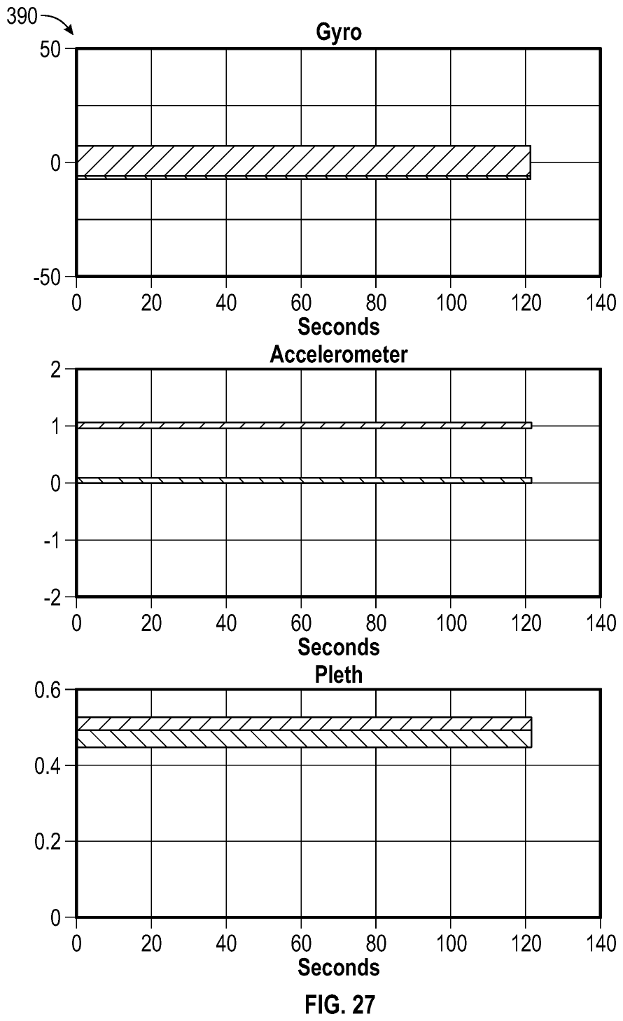

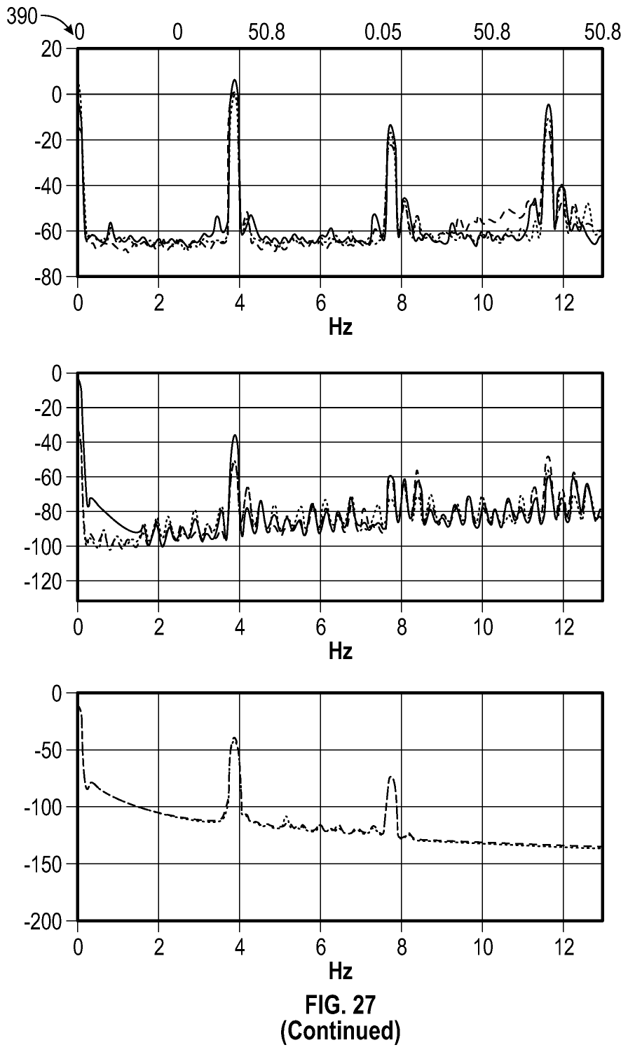

In FIG. 4B, the sensor or module 2700 can include a different arrangement of emitter and detector chambers. The emitter and detector chambers may comprise one or more light blocks. The chambers illustrated in FIG. 27E include a first emitter chamber 2736a enclosing a first emitter group, a second emitter chamber 2736b enclosing a second emitter group, a first group of detector chambers 2740, a second group of detector chambers 2742, and a third group of detector chambers 2738. Each detector chamber can enclose one detector. The first and second emitter chambers 2736a, 2736b can be adjacent to each other. The first, second and third groups of detector chambers 2740, 2742, 2738 can extend around the first and second emitter chambers 2736a, 2736b.

As shown in FIG. 4B, light from the first and second emitter groups in the first and second emitter chambers, respectively, can emit light that travel paths of different lengths for example to different detectors. Light from the first emitter group can travel a shorter path, as indicated by the shorter arrows, to the first group of detector chambers 2740; and light from the first emitter group can travel an intermediate path, as indicated by the intermediate arrows, to the second group of detector chambers 2742; and light from the first emitter group can travel a longer path, as indicated by the longer arrows, to the third group of detector chambers 2738. The reverse is true for light from the second emitter group, which can travel a shorter path to the third group of detector chambers 2738, and an intermediate path to the second group of detector chambers 2742, and a longer path to the first group of detector chambers 2740.

As described herein, the different emitters can be run independently and/or simultaneously. For example, the emitters can be selectively activated (e.g., modulated) so that only one emitter (or subset of emitters) is emitting light at a given time. For example, in embodiments wherein the first emitter group comprises four emitters, each of the four emitters of the first emitter group may be activated for a quarter cycle (e.g., a different quarter cycle than the other emitters) and off for the remaining three-quarters cycle. For example, a first emitter of the first emitter group may be activated to emit light during only a first quarter cycle, a second emitter of the first emitter group may be activated to emit light during only a second quarter cycle, a third emitter of the first emitter group may be activated to emit light during only a third quarter cycle and a fourth emitter of the first emitter group may be activated to emit light during only a fourth quarter cycle. The emitters of the second emitter group may operate in a similar manner as described.

As another example, in embodiments wherein the first emitter group comprises four emitters, each of the four emitters of the first emitter group may be activated for an eighth of a cycle (e.g., a different eighth of the cycle than the other emitters) and off for the remaining seven-eighths cycle. An eighth of a cycle wherein no emitter is activated may occur between each of the cycles wherein an emitter is activated. For example, a first emitter of the first emitter group may be activated to emit light during only a first quarter cycle, a second emitter of the first emitter group may be activated to emit light during only a third quarter cycle, a third emitter of the first emitter group may be activated to emit light during only a fifth quarter cycle and a fourth emitter of the first emitter group may be activated to emit light during only a seventh quarter cycle. The emitters of the second emitter group may operate in a similar manner as described.

The above examples are not meant to be limiting. Alternative activation sequences for the emitters may be used to provide a time-multiplexed signal. In some embodiments, the emitters can be selectively activated (e.g., modulated) so that two or more emitters are emitting light at a given time (e.g., during the same cycle or during overlapping cycles), for example in a manner similar to the examples given above.

The emitters may be modulated within an emitter group (e.g., first emitter group and second emitter group) or all of the emitters of the wearable device 10 may be modulated according to a single activation sequence. For example, the emitters of the first group may be modulated according to one activation sequence and the emitters of the second group may be modulated according to a second activation sequence. Alternatively, the emitters of the first and second emitters groups can all be modulated according to a single activation sequence.

In some embodiments, the detectors may operate independently from and/or simultaneously with each of the other detectors. For example, each of the detectors may provide an individual signal to the module processor 108.

Signals outputted by the different detectors of the different detector chambers 2740, 2742, 2738 based on light emitted from the first emitter group and/or the second emitter group can provide different information due to the different light paths, which can travel through different areas of the tissue. The longer path penetrates deeper into the tissue and through a greater volume of the tissue to reach the detectors of the “far” group of detector chambers than the intermediate and shorter paths. The shorter path penetrates less deep into the tissue and travels through a smaller volume of tissue to reach the detectors of the “near” group of detector chambers than the intermediate and longer paths. The different information can be separated and/or combined to calculate a plurality of physiological parameters of the wearer of the sensor or module 2700, for example, an indication of the wearer's hydration status, which will be described in greater detail below.

For convenience, the terms “proximal” and “distal” are used herein to describe structures relative to the first emitter group or the second emitter group. For example, a detector may be proximal or distal to the first emitter group and may be proximal or distal to the second emitter group. The term “distal” refers to one or more detectors that are farther away from an emitter group than at least some of the other detectors. The term “proximal” refers to one or more detectors that are closer to an emitter group than at least some of the other detectors. The term “intermediate detector” refers to detectors that are closer to an emitter group than distal detectors and farther from an emitter group than proximal detectors. The term “proximal detector” may be used interchangeably with “near detector” and the term “distal detector” may be used interchangeably with “far detector”.

A single detector may be both a proximal to one detector and distal to another detector. For example, a detector may be a proximal detector relative to the first emitter group and may be a distal detector relative to the second emitter group.

As shown in FIGS. 4C and 4D, on the second side of the PCB 2716, which faces away from the cover, the PCB 2716 can be covered by melt plastic or other suitable electronics protective material 2730 (similar to the protective material 130 disclosed herein) except that a flex connector 2732 can remain exposed. The flex connector 2732 can be configured to connect the module 2700 electrically to the wearable device incorporating the module 2700.

### Example Pulse Oximetry Environment

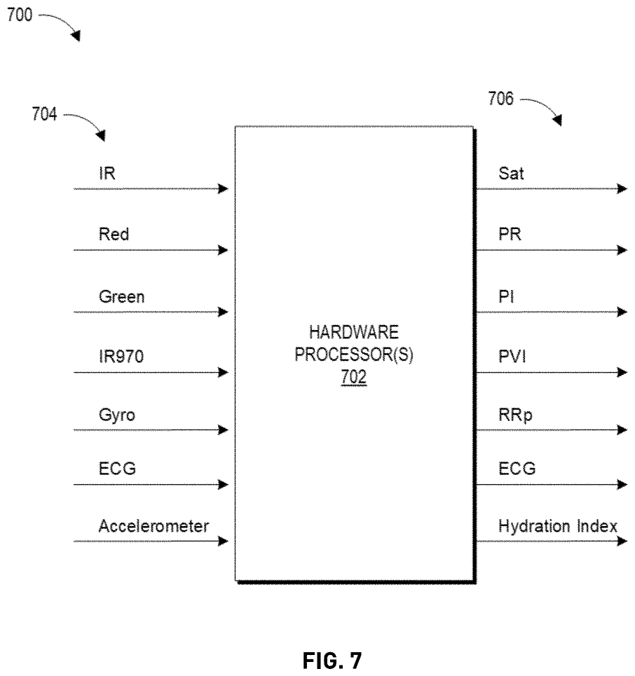

FIG. 7 shows a block diagram 700 of an example sensor device, such as a wearable device. In some examples, the wearable device may include a smart watch. As illustrated in FIG. 7, one or more hardware processors 702 of a wearable device may be configured to process one or more input signals 704. The one or more input signals may include, but are not limited to one or more signals from one or more sensors, such as optical sensors, motion based sensors, or other types of sensors. For example, the one or more hardware processors 702 may be configured to process one or more infrared signals (for example, one or more optical signals at approximately 905 nm or 970 nm, range 800-1,000 nm), red signals (for example, one or more optical signal at approximately 620 nm or 660 nm, range 600-800 nm), green signals (for example, one or more optical signal at approximately 525 nm, range 500-600 nm). Additionally or alternatively, the one or more hardware processers 702 may be configured to processes one or more gyroscope signals, accelerometer signals, or other motion based sensor signals. Additionally or alternatively, the one or more hardware processors 702 may be configured to process an ECG signal or other sensor signal.

In some examples, the one or more hardware processors 702 may be configured to output an output value 706, which may include, but is not limited to one or more physiological parameters or processed signals. In some examples, one or more physiological parameters may include one or more of an SpO2 value, pulse rate (PR), perfusion index (PI), pulse variability index (PVI), respiration rate (RRp), ECG, and hydration index.

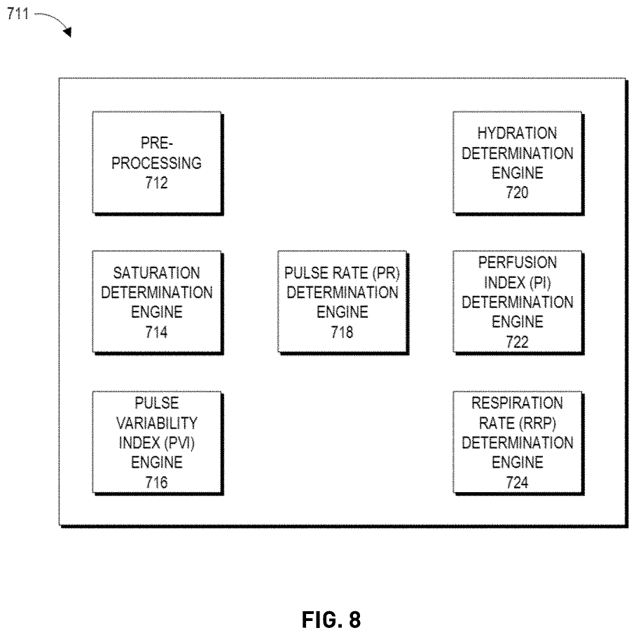

FIG. 8 illustrates an example controller 711 that may be configured to processes the one or more input values 704 to produce the one or more outputs 706. In some examples, the controller 711 may include the one or more hardware processors 702, such as described with reference to FIG. 7. In some examples, a controller 711 may include a pre-processing engine 712, saturation determination engine 714, pulse variability index (PVI) engine 716, pulse rate (PR) determination engine 718, hydration determination engine 720, perfusion index (PI) determination engine 722, and/or a respiration rate (RRp) determination engine 724.

A pre-processing engine 712 can include one or more processes for analyzing, cleansing, editing, reducing, wrangling, or otherwise processing data, such as the one or more input signals 704 for further processing or analysis by one or more engines of the controller 711. In some examples, a pre-processing engine 712 may include one or more processes for cleaning up data using an interpolative process, such as described below with reference to FIG. 27.

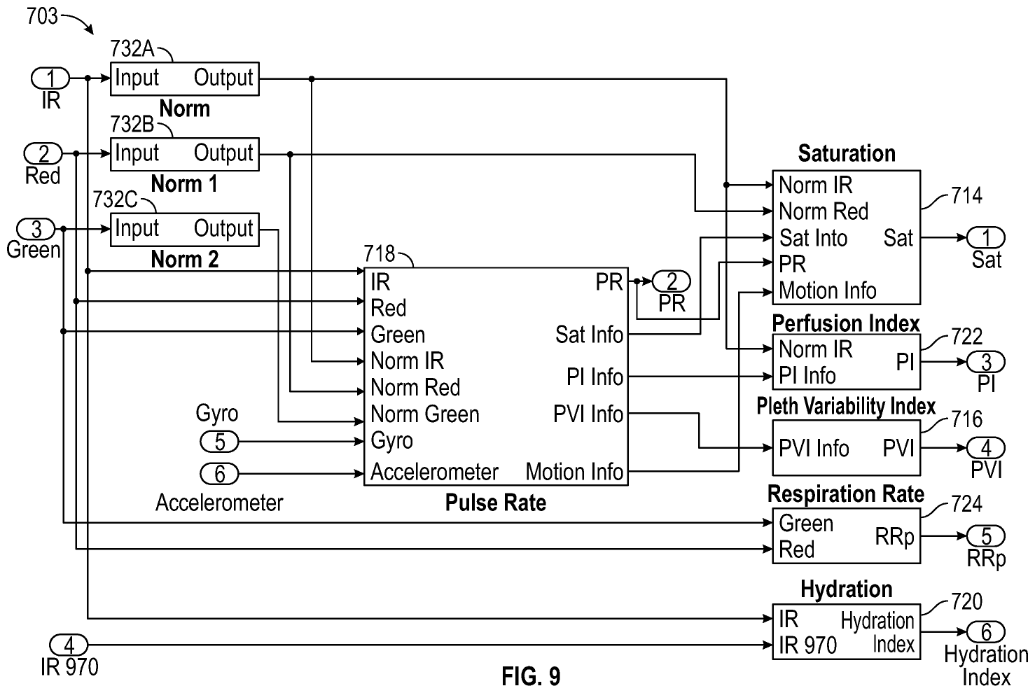

FIG. 9 illustrates example data processing 703 and shows an example of how one or more engines of a controller 711 may interact to produce one or more outputs 706, such as illustrated in FIG. 7. In the illustrated example, one or more optical signals may be normalized or otherwise processed. For example, a controller 711 may normalize an IR signal at a block 732A to produce a normalized IR signal. In another example, a controller 711 may normalize a Red signal at a block 732B to produce a normalized Red signal. In another example, a controller 711 may normalize a Green signal at a block 732C to produce a normalized Green signal. In another example, a controller 711 may normalize an IR970 signal (not shown) to produce a normalized IR970 signal. In some examples, a controller 711 may perform other processing or pre-processing on one or more of the input signals 704. In some examples, a controller 711 may normalize a signal by taking a log of the signal. Other types of data transformation may also be used.

In some examples, a controller 711 may determine a pulse rate using a pulse rate engine 718 using some combination of one or more input signals 704 and/or processed input signals, such as a normalized IR, normalized red, or normalized green signal. In some examples, a pulse rate determination engine 718 may process an IR signal, Red signal, Green signal, normalized IR signal, normalized red signal, normalized green signal, gyroscope signal, accelerometer signal or other input. In some examples, the pulse rate determination engine 718 may output a pulse rate value, blood oxygen saturation (SpO2) information, perfusion index (PI) information, pulse variability index (PVI) information, motion information, one or more confidence values associated with the output, or other output.

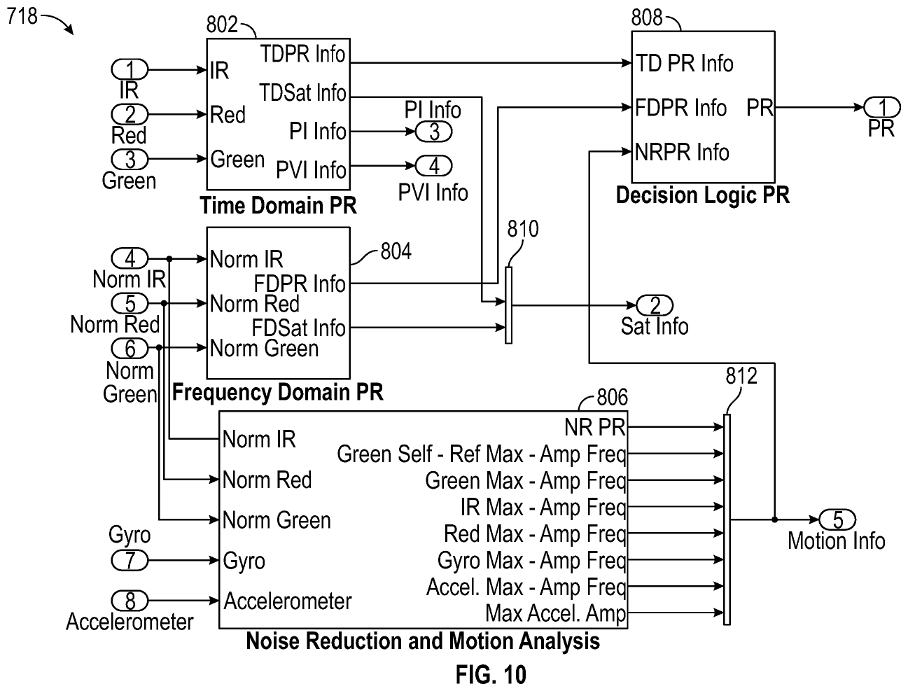

In some examples, a controller 711 may determine an SpO2 value using a saturation engine 714 using some combination of one or more input signals 704, processed input signals, and/or information from another engine of the controller 711. In some examples, a saturation engine 714 may process a normalized IR signal, a normalized red signal, pulse rate, saturation information (determined, for example, using a pulse rate determination engine), motion information (determined, for example, using a pulse rate determination engine), or other input. In some examples, the saturation engine 714 may output a saturation or SpO2 value, a confidence value associated with the output value or other output. In some examples, saturation information may include some combination of frequency domain and/or time domain data associated with one or more input signals, such as described with reference to FIG. 10. In some examples, motion information may include information associated with motion of the user or device, including but not limited to motion features, type of motion (for example, periodic, random), motion rate, magnitude of a gyroscope signal, magnitude of an accelerometer signal, maximum acceleration, or other motion related information.

In some examples, a controller 711 may determine a perfusion index (PI) using a perfusion index engine 722 using some combination of one or more input signals 704, processed input signals, and/or information from another engine of the controller 711. In some examples, a perfusion index engine 722 may process a normalized IR signal and PI information (determined, for example, using a pulse rate determination engine 718). In some examples, the perfusion index engine 722 may output a perfusion index (PI) or PI related value, confidence value, or other output.

In some examples, a controller 711 may determine a pleth variability index (PVI) using a pleth variability index (PVI) engine 716 using some combination of one or more input signals 704, processed input signals, and/or information from another engine of the controller 711. In some examples, a PVI engine 716 may process pleth variability index (PVI) information (determine, for example, using a pulse rate determination engine 718). In some examples, a PVI engine may output a PVI or PVI related value, confidence value, or other output.

In some examples, a controller 711 may determine a respiration rate value (such as RRp) using a respiration rate engine 724 using some combination of one or more input signals 704, processed input signals, and/or information from another engine of the controller 711. In some examples, a respiration rate engine 724 may process a green signal and a red signal. In some examples, a respiration rate engine 724 may output a respiration rate or respiration rate related value, confidence value, or other output.

In some examples, a controller 711 may determine a hydration index using a hydration engine 720 using some combination of one or more input signals 704, processed input signals, and/or information from another engine of the controller 711. In some examples, a hydration engine 720 may process an infrared signal and an IR970 (for example, a signal at approximately 970 nm). In some examples, a hydration engine 720 may output a hydration index or hydration related value, confidence value, or other output.

### Example Pulse Rate Determination

In some instances, when processing the detected signals of the attenuated light after absorption by a tissue site of the wearer from the detectors 106, 306a, 306b, the plethysmograph module 100, 300 can reduce noise in these detected signals. For example, the plethysmograph module 100, 300 can use a noise reference signal to reduce the noise in the other detected signals. As discussed above, the emitters 104, 304a, 304b of the plethysmograph module 100, 300 can emit light corresponding to multiple wavelengths, such as a first, second, third, and/or fourth wavelength. The first wavelength can be from about 525 nm to about 580 nm and the light can correspond to a green spectrum. The first wavelength can provide the reference signal, which can be stronger and less likely to be affected by noise than the detected signals generated from the other wavelengths. The second wavelength can be from about 620 nm to about 660 nm and the light can correspond to a red spectrum. The light corresponding to the second wavelength can be more sensitive to changes in blood oxygen saturation, SpO2. The third wavelength can be about 900 nm to about 910 nm and the light can correspond to an infrared spectrum.

The detectors 106, 306 of the plethysmograph module 100, 300 may have a noise floor such that the detected signals generated in response to the second wavelength, or other wavelengths, interacting with the tissue site may be more effected by noise or more susceptible to errors than the detected signals generated in response to the first wavelength, or other wavelengths, interacting with the tissue site. Therefore, the noise reference signal can be used by the plethysmograph module processor 108, 308 to remove noise from the plethysmograph waveform produced by the detected signals generated from the second wavelength, which can be close to the noise floor of the detector 106, 306. For example, the noise reference signal can be used to subtract noise from the plethysmograph waveform(s) produced by the detected signals generated in response to the second wavelength and/or the third wavelength interacting with the tissue site. Light corresponding to the first wavelength can be selective for the wearer's pulse rate such that the plethysmograph waveform produced by the detected signals generated in response to the first wavelength interacting with the tissue site correlates with the wearer's pulse rate. The plethysmograph module 100, 300 can identify the peaks in the pleth waveform based on the detected signals generated from the first wavelength and, using the pleth waveforms based on the detected signals from the second and third wavelengths, determine the wearer's SpO2 at these points.

Additionally, the wearer's movements (e.g., walking, jogging) may also affect the plethysmograph module's 100, 300 determination of the wearer's SpO2 by causing noise in the detected signals generated from the second wavelength and/or third wavelength. The plethysmograph module processor 108, 308 can use the gyroscope 112 and/or the accelerometer 114 to make further corrections to the pleth waveform(s) based on the detected signals generated from the second wavelength and/or the third wavelength to reduce noise. For example, signals from the gyroscope 112 and/or the accelerometer 114 can be used to identify and remove peaks in the pleth waveform(s) that are attributable to the wearer's movement rather than the wearer's pulse rate.

A controller 711 may utilize a green signal in order to determine a pulse rate. A green signal may be advantageous in determining a pulse rate from a wrist due to its increased strength or signal quality over an infrared or red signal. In some examples, a controller 711 may use the green signal in order to correct the placement of an IR/Red ratio for pulse oximetry. In some examples, a green signal may be used to reduce noise in an optical signal. In some examples motion analysis using one or more motion based sensors (such as a gyroscope and/or accelerometer signal) may be used to reduce noise in an optical signal.

In some examples, a pulse rate determination engine 718 may include a plurality of individual pulse rate engines. FIG. 10 illustrates an example pulse rate determination engine 718. For example, a pulse rate determination engine 718 may include a time domain pulse rate engine 802, a frequency domain pulse rate engine 804, and a noise reduction pulse rate engine 806.

In some examples, calculated values from one or more of the engines may be output or bundled for further processing. For example, a pulse rate determination engine 718 may output a determined pulse rate, PI information, PVI information, saturation information 810 (such as a saturation information determined using a time domain pulse rate engine 802 and/or saturation information determined using a frequency domain pulse rate engine 804), motion information 812 (such as some combination of green signal self-reference signal maximum amplitude frequency, green signal maximum amplitude frequency, infrared signal maximum amplitude frequency, red signal maximum amplitude frequency, red signal maximum amplitude frequency, gyroscope signal maximum amplitude frequency, accelerometer signal maximum amplitude frequency, and maximum acceleration signal amplitude).

A time domain pulse rate engine 802 may include at least one process or algorithm for determining a pulse rate in the time domain using one or more input signals, that may include, but are not limited to, an IR signal, Red signal, Green signal, and IR970 signal. Because green can be a stronger signal than Red or IR, a green signal may be used as a primary signal for determination of a good candidate pulse rate or pleth signal. In some examples, when a controller 711 determines a satisfactory candidate pulse rate in the green signal, the controller 711 may select or determine a corresponding pleth signal in the IR and Red. A corresponding pleth signal may include a red and/or IR signal at or near the same time stamp as the candidate pulse in the green signal. A Red/IR ratio may then be determined in order to estimate saturation. A green candidate pulse may be determined based on one ore more similar or different processes to select a Red or IR candidate pulse. In some examples, aspects of candidate pulse selection associated with Red or IR signals may not be performed for a green candidate pulse, such as the application of a stick error model. In some examples, a time domain pulse rate engine 802 may have the same or different pre-processing of signals, such as described in U.S. Pat. No. 5,632,272, titled “SIGNAL PROCESSING APPARATUS,” issued May 27, 1997, U.S. Pat. No. 6,002,952, titled “SIGNAL PROCESSING APPARATUS AND METHOD,” issued Dec. 14, 1999, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,463,311, titled ‘PLETHYSMOGRAPH PULSE RECOGNITION PROCESSOR,” issued Oct. 8, 2002, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein. For example, pre-processing may or may not include a stick model check. In some examples, the stick model check may be less stringent. For example, a stick model error in a green candidate pulse may be associated with a higher threshold than in with a red or IR candidate pulse. In some examples, a cross-correlation check may be different and/or updated for the use of a green signal. For examples, a cross-correlation check may determine whether there is satisfactory correlation for all channel signals, green, red, and IR before moving on to the next processing stage. In some examples, an output of a time domain pulse rate engine 802 may include a time domain pulse rate, time domain saturation information, PI information, and PVI information.

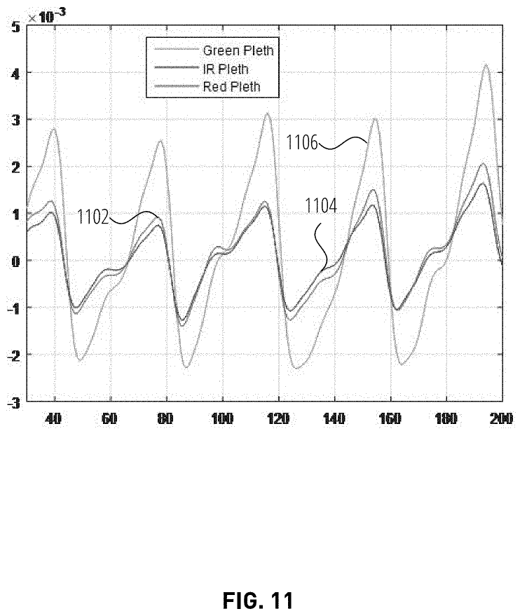

A frequency domain pulse rate engine 804 may include at least one process or algorithm for determining a pulse rate in the frequency domain using one or more input signals, that may include, but are not limited to, a normalized IR signal, a normalized Red signal, a normalized Green signal, and a normalized IR970 signal. Similar to described above with reference to the time domain pulse rate engine 802, a green signal may be used as a primary signal for determination of a candidate pulse rate or pleth signal due to it being potentially stronger than Red or IR signal. In some examples, when a controller 711 determines a satisfactory candidate pulse rate in the green signal, the controller 711 may select or determine a corresponding pleth signal in the IR and Red. A corresponding pleth signal may include a red and/or IR signal at or near the same time stamp as the candidate pulse in the green signal. FIG. 11 illustrates an example set of signals corresponding to a red signal 1002, IR signal 1004, and pleth signal 1106.

A Red/IR ratio may be determined in order to estimate saturation. In some examples, a frequency domain pulse rate engine 802 may have the same or different pre-processing of signals, such as described in U.S. Pat. No. 5,632,272, titled “SIGNAL PROCESSING APPARATUS,” issued May 27, 1997, U.S. Pat. No. 6,002,952, titled “SIGNAL PROCESSING APPARATUS AND METHOD,” issued Dec. 14, 1999, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,463,311, titled ‘PLETHYSMOGRAPH PULSE RECOGNITION PROCESSOR,” issued Oct. 8, 2002, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein. In some examples, a frequency domain pulse rate engine 804 may use the spectrum derived from green PPG signal in addition to or instead of an IR PPG signal for frequency domain analysis and/or pulse rate determination. In some examples, a signal reference spectrum in a frequency domain pulse rate engine 804 may be based on one or more spectrums derived from green and/or red PPG signals instead of or in addition a to spectrums derived from IR and/or red PPG signals. In some examples, one or more thresholds associated with the frequency domain pulse rate engine 804 may be related to a range of Red/IR ratios. For example, wavelength of a red signal may be 620 nm instead of 660 nm at 100 percent SpO2 and so a 620 nm/IR ratio is approximately 1 while a 660 nm/IR ratio would be approximately 0.5. In some examples, an output of a frequency domain pulse rate engine 804 may include a frequency domain pulse rate, and frequency domain saturation information.

A noise reduction pulse rate engine 806 may include at least one process or algorithm for determining a pulse rate using one or more processes for noise reduction or interference mitigation. In some examples, a noise reduction pulse rate engine 806 may include one or more processes for analyzing motion of a user, sensor, and/or device. The noise reduction pulse rate engine 806 may determine a pulse rate and/or information related to the motion of the user, sensor, and/or device using inputs that may include, but are not limited to, a normalized infrared signal, a normalized red signal, a normalized green signal, a gyroscopic signal, and an accelerometer signal.

In some examples, a noise reduction or interference mitigation process of a noise reduction pulse rate engine 806 may include one or more processes for cleaning up a signal, such as a Green PPG signal, Red PPG signal, or IR PPG signal. In some examples, a controller 711 may use one or more of the input signals or values in order to generate a noise or signal reference for use in cleaning up a signal. For example, a controller 711 may generate or use a noise reference based on some combination of gyroscope axis signals, some combination of accelerometer axis signals, an integral of at least one accelerometer axis (which may correspond to a velocity value), a double integral of at least one accelerometer axis (which may correspond to a position value), an integral of at least one gyroscope axis, an infrared PPG signal, a red PPG signal, some combination of gyroscope axis and accelerometer axis signals as multiple noise references, Green PPG signal, the like or a combination thereof. In some examples, noise cancellation may be done in FFT space.

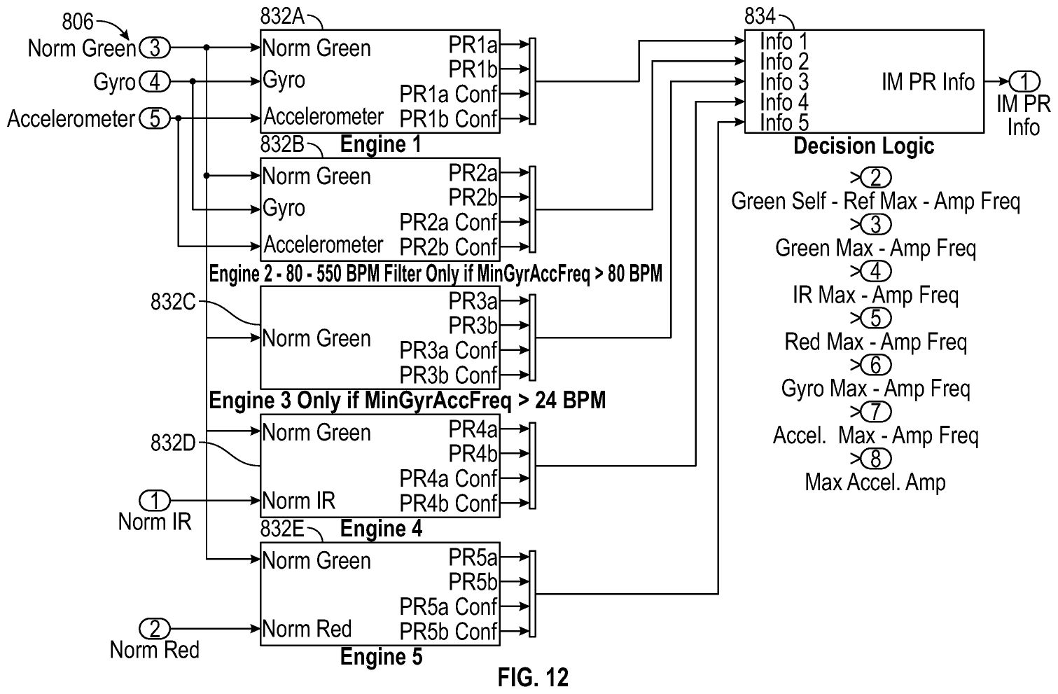

In some examples, such as illustrated in FIG. 12, a noise reduction pulse rate engine 806 may include a plurality of noise reduction sub-engines or processes 830A, 830B, 830C, 830D, 830E and noise reduction decision logic 832. One or more of the noise reduction sub-engines 830A, 830B, 830C, 830D, 830E may be configured to provide a pulse rate estimate. In some examples, the noise reduction decision logic 832 may be configured to select a candidate estimate from the one or more pulse rate estimates provided by the one or more noise reduction sub-engines 832A, 832B, 832C, 832D, 832E. It is of note that while five sub-engines are illustrated in FIG. 12, there may be more or fewer noise reduction sub-engines.

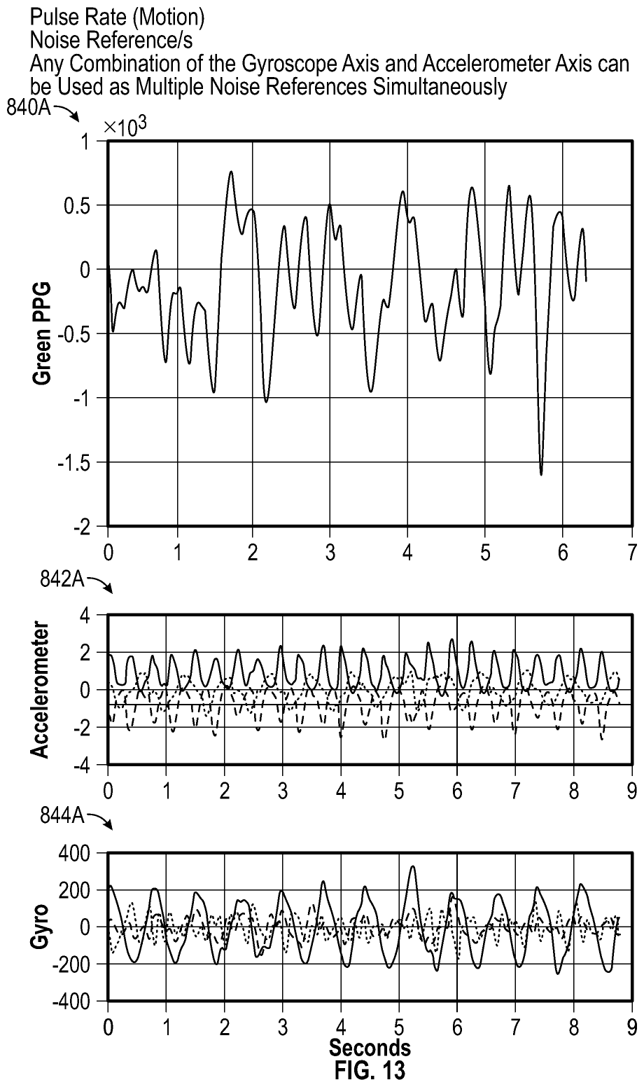

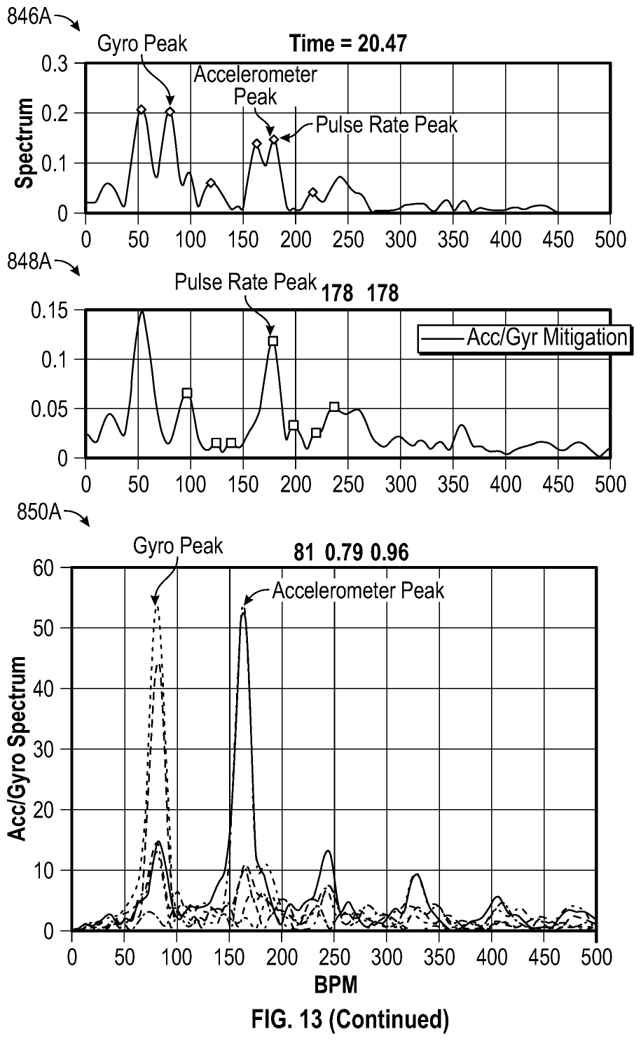

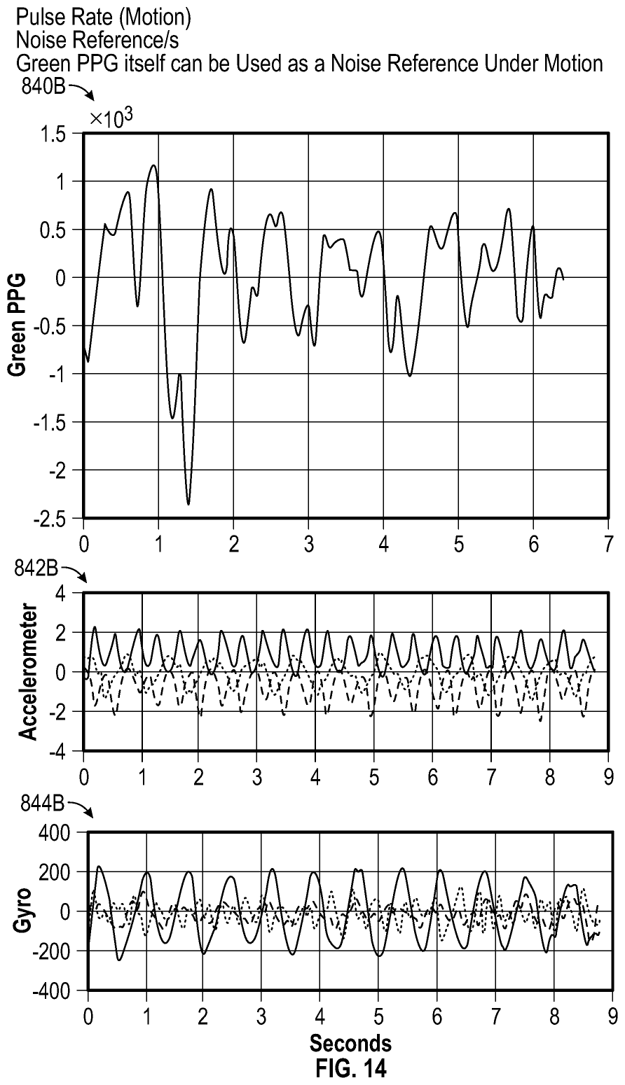

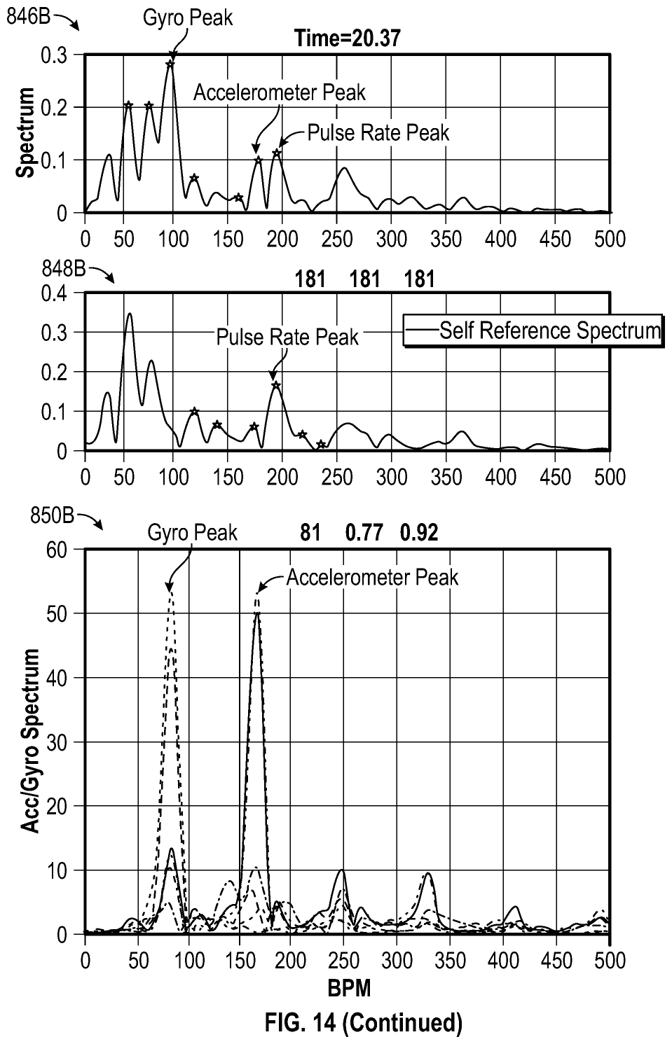

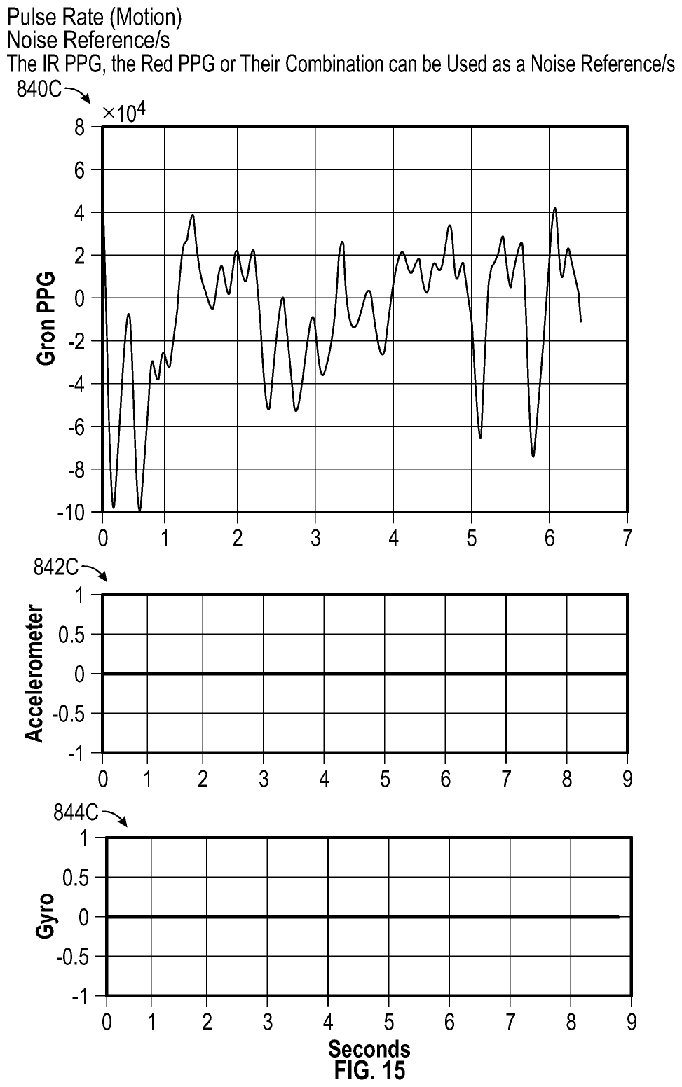

In some examples, a noise reduction engine 806 may include a pulse rate estimate sub-engine 832A configured to determine a candidate pulse rate using green PPG signals and one or more of a gyroscope and acceleration signal as one or more noise references. FIG. 13 illustrates example green PPG signals 840A, accelerometer signals 842A, gyroscope signals 844A that may be received as input. A frequency domain representation of a green signal 846A illustrates a plurality of peaks corresponding to a peak associated with the gyroscope signal, a peak associated with the accelerometer signal, and a pulse rate peak. As part of a pulse rate estimate sub-engine 832A may remove or reduce peaks associated with the gyroscope and/or the accelerometer, such as illustrated in graph 850A to generate a noise reduced signal 848A. In some examples, the axis having the largest magnitude of the accelerometer and/or gyroscope signal may be used as representative of the motion signal for use in noise reduction. However, other processing and/or use of the accelerometer and/or gyroscope signals may be used in addition or in the alternative. A controller 711 may then determine at least one candidate pulse rate and/or at least one confidence score associated with the candidate pulse rate(s) based on the noise reduced signal 848A. In some examples, a controller 711 may determine two candidate pulse rates and two confidence scores.

In some examples, a noise reduction engine 806 may include a pulse rate estimate sub-engine 832B configured to determine a candidate pulse rate using a filtered green PPG signals and one or more of a gyroscope and acceleration signal as one or more noise references. In some examples, a controller 711 may apply a bandpass filter to generate a filtered green PPG signal. In some examples, the bandpass filter may select signal between 80 and 550 BPM or another range that is greater or smaller than between 80 and 550 BPM. In some examples, the pulse rate sub-engine 832B may be configured to determine a pulse rate estimate if a condition is met. For example, a condition may include a minimum frequency in one or more of a gyroscope and acceleration signal. In some examples, the axis having the largest magnitude of the accelerometer and/or gyroscope signal may be used as representative of the motion signal for use in noise reduction. However, other processing and/or use of the accelerometer and/or gyroscope signals may be used in addition or in the alternative. In some examples, a minimum frequency may include 80 BPM or another frequency. In some examples, the minimum frequency may correspond to a lower limit of a bandpass filter frequency. In some examples, the lower limit frequency may correspond to an estimated minimum frequency associated with a gyroscope and/or acceleration signal. In some examples, this estimated minimum frequency is 80 beats per minute (BPM), however, other values are also possible. 80 BPM may be used as a threshold minimum frequency because, for example, when a minimum accelerometer or gyroscope frequency is at or around 80 BPM, a pulse rate of a user may be high. Based on this assumption, the pleth can thus filtered to remove low frequencies (such as those less than 80 BPM) and noise cancellation applied to the filtered pleth to then estimate a candidate pulse rate. It is of note that while 80 BPM can be used as a threshold minimum frequency, other threshold frequencies may additionally or alternatively be used. For example, a threshold minimum frequency of 60, 70, 90, 100 or different BPM may be used. Advantageously, the filtering may allow a controller 711 to preliminarily remove peaks in signal that are less likely to be associated with a pulse rate because a pulse rate is likely to be greater than the minimum frequency associated with a motion signal. For example, a user's pulse rate may be estimated to be greater than their walking pace. In some examples, the estimated minimum frequency may be determined based on a relationship between one or more of the input signals, such as the gyroscope and accelerometer signals. For example, if a gyroscope signal is double or half of an accelerometer signal, the minimum of the two signals may be associated with the minimum threshold for pulse rate and/or lower limit of a filter. In some examples, if an accelerometer and gyroscope signal are similar (for example, contain a threshold amount of overlap), a controller 711 may determine a minimum threshold of half of the accelerometer and/or gyroscope signal frequency. It is of note that the accelerometer and/or gyroscope signal may overlap during walking or other forms of movement. A controller 711 may determine at least one candidate pulse rate and/or at least one confidence score associated with the candidate pulse rate(s). In some examples, a controller 711 may determine two candidate pulse rates and two confidence scores.

In some examples, a noise reduction engine 806 may include a pulse rate estimate sub-engine 832C configured to determine a candidate pulse rate using a green PPG signal as a self-reference. For example, the green PPG signal may be offset to generate an offset sample. The offset sample may be subtracted from the green PPG signal. In this way, the offset sample may be treated as noise and the non-offset sample may be treated as signal plus noise. In some examples, the offset sample may be offset by a period of motion. The self-reference noise reduction is based on an assumption that the pulse rate is independent of motion. Thus, when the signal is offset by a period of motion and subtracted from the non-offset signal, the motion period will align and be cancelled out, while the pulse rate will not. Thus, the resulting signal will be primarily indicative of a pulse rate and have reduced effects from periodic motion. In some examples, the self-reference sub-engine 832C may be applied if the minimum gyroscope and/or accelerometer frequency exceeds a minimum threshold. In some examples, the axis having the largest magnitude of the accelerometer and/or gyroscope signal may be used as representative of the motion signal for use in noise reduction. However, other processing and/or use of the accelerometer and/or gyroscope signals may be used in addition or in the alternative. The minimum threshold may include, but is not limited to, 24 BPM. In some examples, a minimum pulse rate estimated by the controller may be 25 BPM. Thus, if the accelerometer and/or gyroscope frequencies are below 24 BPM, those noise frequencies may show up on a pleth signal. Filters may remove frequencies below 25 BPM from the pleth signal. So lower frequencies will thus not affect pulse rate estimation. If the noise frequencies are below 24 BPM, then the filter will, then the controller may not need to apply adaptive noise cancellation in order to remove those noise frequencies from the signal. Conditions where the minimum gyroscope and/or accelerometer frequency fall above the determined minimum may correspond to situations where the green PPG signal is more likely to be accurate.

FIG. 14 illustrates example green PPG signals 840B, accelerometer signals 842B, gyroscope signals 844B that may be received as input. A frequency domain representation of a green signal 846B illustrates a plurality of peaks corresponding to a peak associated with the gyroscope signal, a peak associated with the accelerometer signal, and a pulse rate peak. As part of a pulse rate estimate sub-engine 832C may use a green PPG signal as a self-reference, such as described above to generate a noise reduced signal 848B. The self-reference process may reduce peaks associated with a gyroscope and accelerometer, such as illustrated in graph 850B. A controller 711 may then determine at least one candidate pulse rate and/or at least one confidence score associated with the candidate pulse rate(s) based on the noise reduced signal 848B. In some examples, a controller 711 may determine two candidate pulse rates and two confidence scores.

With continued reference to FIG. 12, in some examples, a noise reduction engine 806 may include a pulse rate estimate sub-engine 832D to determine a candidate pulse rate using a green PPG and an IR PPG signal. For example, a controller 711 may use an IR signal as a noise reference and subtract the noise reference from the green PPG signal to generate a noise reduced signal.

In some examples, a noise reduction engine 806 may include a pulse rate estimate sub-engine 832E to determine a candidate pulse rate using a green PPG and a red PPG signal. For example, a controller 711 may use a red signal as a noise reference and subtract the noise reference from the green PPG signal to generate a noise reduced signal.

FIG. 15 illustrates example green PPG signals 840C, accelerometer signals 842C, gyroscope signals 844C that may be received as input. As illustrated, an accelerometer signal 842C and gyroscope signal 844C may be below a threshold amplitude or not received such as shown in graph 850C. A frequency domain representation 846C of a green PPG signal 852A, IR PPG signal 852B, and red PPG signal 852C illustrates a plurality of peaks corresponding to one or more peaks associated with noise and a pulse rate peak. As part of a pulse rate estimate sub-engine 832D or 832E may use a red and/or IR PPG signal as a noise reference, such as described above to generate a noise reduced signal 854A or 854B. The process may reduce peaks associated with noise. A controller 711 may then determine at least one candidate pulse rate and/or at least one confidence score associated with the candidate pulse rate(s) based on the noise reduced signal 854A or 854B. In some examples, a controller 711 may determine two candidate pulse rates and two confidence scores for each sub-engine.

While certain noise reference signals are discussed above, other noise reference signals may be used in addition or in the alternative to those discussed above. For example, some signals may be combined as a noise reference, such as a red and IR PPG signal or gyroscope and accelerometer signal. In some examples, a processed version of one or more input signals may be used as a noise reference, such as an nth order derivative or integral of a signal. In some examples, an integral of an accelerometer axis, which may correspond with velocity, or a double integral of an accelerometer axis, which may correspond with position, may be used as a noise reference. In some examples, the integral of a gyroscope axis can be used as a noise reference. In some examples, a noise estimate signal may be used as a noise reference, such as a sine wave or other periodic signal. In some examples, a controller 711 may use noise cancellation in the FFT space.

In some examples, one or more of the sub-engines may be configured to select one or more candidate pulses having the highest confidence using one or more selection processes. For example, a sub-engine may be configured to select a first candidate pulse using a first confidence determination and a second candidate pulse using a second confidence determination. In some examples, the first and second candidate pulses may be the same. In some examples, the first and second candidate pulses may be different.

In some examples, in a first confidence determination process, a controller 711 may assign a higher confidence score to a peak with a higher magnitude. For example, a peak with the highest magnitude may be the peak with highest confidence. In some examples, a peak magnitude may be determined by calculating a sum of harmonics associated with the peak in a sampled signal divided by the total energy in the sampled signal.

In addition or in the alternative to the first confidence determination process, in a second confidence determination process, a controller 711 may make a confidence determination using a different method than the first confidence determination process. For example, a controller 711 may calculate a parameter based on the sum of harmonics associated with the peak in a sampled signal divided by the total energy of peaks above the analyzing peak. The controller 711 may assign the highest confidence to the peak having the greatest value of that parameter.

With continued reference to FIG. 12, a noise reduction engine 806 may include decision logic 806 configured to select a candidate pulse rate from one or more pulse rates generated by the one or more sub-engines 832A, 832B, 832C, 832D, 832E. In some examples, the controller 711 may determine one or more confidence scores associated with one or more candidate pulse rates from the one or more sub-engines.

In some examples, the decision logic 806 may select a candidate pulse as the output of the noise reduction engine 806 by analyzing and comparing the outputs of the one or more sub-engines. In some examples, the decision logic 806 may use a combination of confidence checks and/or checks if candidates are consistent between sub-engines to identify a candidate pulse rate. In some examples, the decision logic 806 may select a candidate pulse rate based on a preference of a sub-engine. For example, a first sub-engine may be more preferred over a second sub-engine. In some examples, a sub-engine configured to utilize one or more of a gyroscope or accelerometer signal, such as sub-engine 832A or 832B may be preferred over a sub-engine configured to utilize a green and/or IR or Red signal, such as sub-engine 832C, 832D, or 832E. For example, decision logic may make a first check for a candidate pulse from sub-engine 832A, a decision logic may then move on to sub-engine 832B, sub-engine 832C, sub-engine 832D, sub-engine 832E, and continue on through any remaining sub-engines to select a candidate pulse. In some examples, if no satisfactory sub-engine is determined after checking the outputs of the sub-engines, a controller 711 may output zero or a high confidence value for a pulse rate. In some examples, the high confidence value may include a pulse rate determined based on a running average of the pulse rate. For example, the controller 711 may output a pulse rate that falls within a predetermined range of recent values or a running average of a pulse rate, if available.

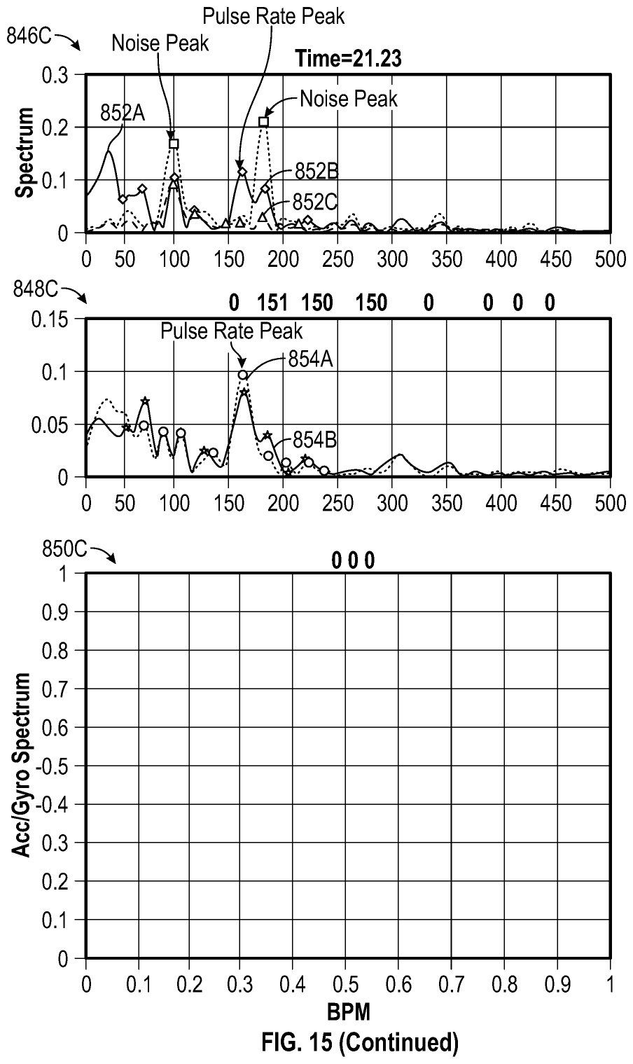

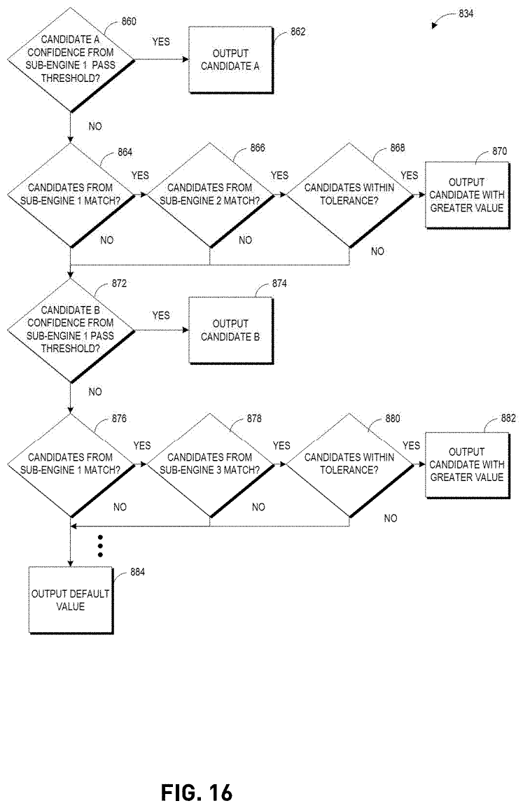

FIG. 16 illustrates example decision logic 806 that may be used to select a candidate pulse rate from a plurality of sub-engines that are configured to produce a two candidate pulse rates and corresponding confidence scores. For example, a controller, at block 860, may check if a confidence score associated with a first candidate pulse rate from a first sub-engine passes a threshold confidence value. In some examples, the confidence threshold value may be determined based on a linear relationship between pulse rate and confidence. If the confidence score passes the threshold, then the controller 711 may be configured to output the candidate pulse rate at block 862.

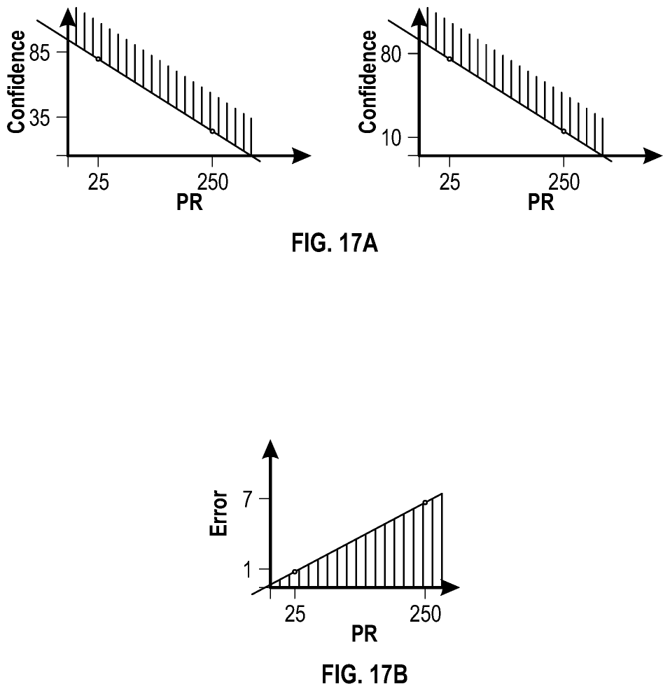

As illustrated in FIG. 17A, a threshold value for confidence may be different based on the value of the candidate pulse rate. For example, a pulse rate of 250 bpm may have a corresponding threshold confidence of 35, whereas a pulse rate of 80 may have a higher confidence, such as a threshold confidence of 75. In some examples, the linear relationship between pulse rate and threshold confidence may be different for different sub-engines. For example, a controller 711 may determine that a threshold confidence for candidate pulse rates associated with a first sub-engine may be different from a threshold confidence for candidate pulse rates associated with a second sub-engine.

With continued reference to FIG. 16, if the confidence score does not pass the threshold, then the controller 711 may determine whether a first and second candidate from a first sub-engine match or are satisfactorily similar at block 864. In some examples, the controller 711 may also determine whether a first and second candidate from a second sub-engine match or are satisfactorily similar at block 866. Candidates may be determined to match if they have the same value or are within a determined percentage of each other, such as a 0.5 percent, 1 percent, 5 percent, 10 percent, or more or less percent. In some examples, if both candidates from the first sub-engine match and both candidates from the second sub-engine match or are substantially similar as determined at block 864 and block 866, the controller 711 may determine whether the candidates from the first sub-engine and the candidates from the second sub-engine fall within an acceptable tolerance of each other at bock 868. In some examples, an acceptable tolerance may include a determination that a difference between a candidate pulse rate value of the first sub-engine and a candidate pulse rate value of the second sub-engine fall below a threshold value. In some examples, a threshold value for a tolerance or error may be different based on pulse rate. For example, the threshold value may be determined based on a linear relationship between pulse rate and confidence. If the candidates are determined to be within a threshold tolerance at block 868, the controller 711 may output a candidate pulse that is the greater of the candidate pulse rate value from the first sub-engine and the candidate pulse rate from the second sub-engine.

As illustrated in FIG. 17B, a threshold value for tolerance or error may be different based on the value of a candidate pulse rate. In some examples, the value of the candidate pulse rate may be the maximum, average, or other value associated with the candidate pulse rate values form the compared sub engines. For example, a candidate pulse rate used in the threshold value determination may be the greater of the candidate pulse rate value from the first sub-engine and the candidate pulse rate from the second sub-engine. In some examples, a pulse rate of 250 bpm may have a corresponding threshold tolerance of 7, whereas a pulse rate of 80 may have a lower error or tolerance, such as a threshold tolerance of 2. In some examples, the linear relationship between pulse rate and threshold confidence may be different for different sub-engines. For example, a controller 711 may determine that a threshold confidence for candidate pulse rates associated with a first sub-engine may be different from a threshold confidence for candidate pulse rates associated with a second sub-engine.

With continued reference to FIG. 16, if the candidates do not match at blocks 864 or block 866, or do not fall within the threshold tolerance at block 868, the controller, at a block 872, may check if a confidence score associated with a second candidate pulse rate from a first sub-engine passes a threshold confidence value. In some examples, the confidence threshold value may be determined based on a linear relationship between pulse rate and confidence. If the confidence score passes the threshold, then the controller 711 may be configured to output the candidate pulse rate at block 874. The threshold confidence may be the same or different as described above with reference to block 862 and FIG. 17A.

In examples, where there are three or more sub-engines, a controller 711 may determine whether a first and second candidate from a first sub-engine match or are satisfactorily similar at block 876 if the confidence score does not pass the threshold. In some examples, the controller 711 may also determine whether a first and second candidate from a third sub-engine match or are satisfactorily similar at block 866. Candidates may be determined to match if they have the same value or are within a determined percentage of each other, such as a 0.5 percent, 1 percent, 5 percent, 10 percent, or more or less percent. In some examples, if both candidates from the first sub-engine match and both candidates from the third sub-engine match or are substantially similar as determined at block 878 and block 876, the controller 711 may determine whether the candidates from the first sub-engine and the candidates from the third sub-engine fall within an acceptable tolerance of each other at bock 880. In some examples, an acceptable tolerance may include a determination that a difference between a candidate pulse rate value of the first sub-engine and a candidate pulse rate value of the third sub-engine fall below a threshold value. In some examples, a threshold value for a tolerance or error may be different based on pulse rate. For example, the threshold value may be determined based on a linear relationship between pulse rate and confidence. If the candidates are determined to be within a threshold tolerance at block 880, the controller 711 may output a candidate pulse that is the greater of the candidate pulse rate value from the first sub-engine and the candidate pulse rate from the third sub-engine. The threshold tolerance may be the same or different as described above with reference to block 868 and FIG. 17B.

One or more of the above mentioned blocks, checks, or processes may be repeated by the controller 711 until an end condition is met, such as where no sub-engine is determined to have a satisfactory candidate pulse rate, a satisfactory candidate pulse rate is selected, or another end condition occurs. In some examples, a controller 711 may perform similar steps for some or all of the sub-engines of a noise reduction engine 806. In some examples, if an end condition is reached and no satisfactory candidate pulse is selected, then the controller 711 may output a default value as a candidate pulse at block 884. The default value may include a zero value or a nonzero value. In some examples, a nonzero value can include an estimated pulse rate, such as a running average pulse rate.

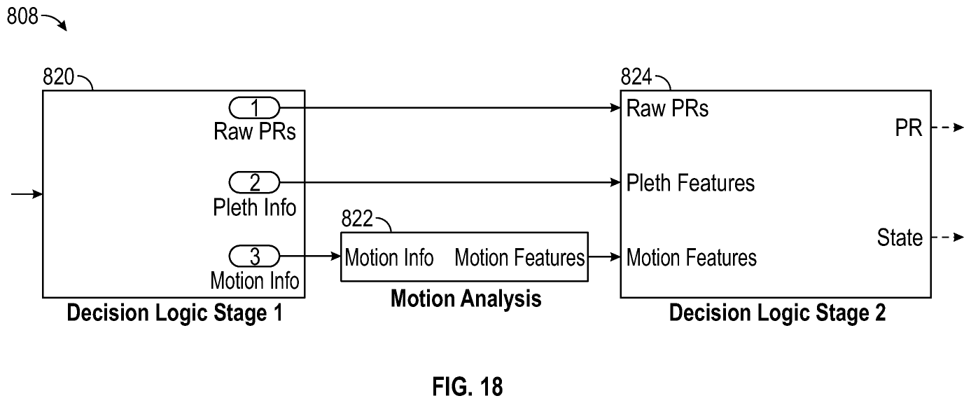

FIG. 18 illustrates aspects of decision logic 808 that may be used to select a candidate pulse rate from one or more of the candidate pulse rates determined by the one or more pulse rate engines, such as engines 802, 804, and 806. For example, decision logic 808 may include a plurality of stages, such as a first stage 820 and a second stage 824. It is of note that while a first stage 820 and a second stage 824 are illustrated as performed in a certain order, the decision logic stages may be performed in any suitable order. In some examples, a controller 711 may process data before, after, or between decision logic stages. For example, as illustrated in FIG. 18, a controller 711 may perform a motion analysis 822 of motion information determined by the controller 711 at an earlier stage.

One or more inputs to the decision logic 808 or aspects of the decision logic 808 may include one or more candidate pulse rates from the one or more pulse rate engines, pleth info, and motion info. In some examples, the one or more candidate pulse rates may include one or more data types, including but not limited to the calculated candidate pulse rates from the individual engines and one or more associated confidence scores associated with the calculated candidate pulse rates. In some examples, pleth info may include one or more data types. For example, pleth info may include time domain and frequency domain features for some or all of the channels of the pleth signal. In some examples, the number of channels may include 4, however more or fewer channels are also possible.

In some examples, the time domain and frequency domain features may include, but is not limited to, maximum peak position (in, for example, beats per minute) in the spectrum across wavelengths or the channels of the signal and correlation of channels. Correlation of channels may include, for example, information associated with how synced the different channels are, such as a correlation of the IR channel or signal and the red channel or signal, correlation of the green channel or signal and the IR channel or signal, and correlation of the green channel or signal and the red channel or signal. A degree of correlation may be categorized into a scale or score. For example, the correlation may be on a scale of 0 to 1, where a low correlation may be closer to 0 and a high correlation may be closer to 1. In some examples, a high correlation across channels, such as red and IR, may indicate low or minimal motion. In another example, a low correlation across channels, such as red and IR, may indicate a lot of motion.

Other pleth information may also be used. In some examples, motion information may include, but is not limited to, information associated with motion of the user or device. For example, motion information may include, but is not limited to motion features, type of motion (for example, periodic, random), motion rate, magnitude of a gyroscope signal, magnitude of an accelerometer signal, maximum acceleration, or other motion related information. In some examples, motion information may include data output from one or more pulse rate engines, such as a noise reduction engine 806. In some examples, motion information may include signal associated with a six degree of freedom inertial measurement unit (IMU).

One or more outputs of the decision logic 808 may include, but is not limited to, a selected candidate pulse rate and a state value. The state value may include, for example, a tracking value associated with the selected candidate pulse configured to illustrate the path (or engine) used to determine the selected candidate pulse. In another example, the state value may additionally or alternatively include a confidence measure. In some examples, the state value may indicate the type of logic, rule, and/or features used to generate a decision. This can help explain why a decision is made by the decision logic 912 and the explanation itself can additionally or alternatively be a feature for analysis.

In some examples, a decision logic stage 820 may include a first pulse rate determination or selection of at least one candidate pulse based on one or more selection processes, such as described in U.S. Pat. No. 5,632,272, titled “SIGNAL PROCESSING APPARATUS,” issued May 27, 1997, U.S. Pat. No. 6,002,952, titled “SIGNAL PROCESSING APPARATUS AND METHOD,” issued Dec. 14, 1999, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,463,311, titled ‘PLETHYSMOGRAPH PULSE RECOGNITION PROCESSOR,” issued Oct. 8, 2002, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein. In some examples, the decision logic stage 820 may serve to determine pleth quality prior to further analysis of the candidate pulse rates by the controller. For example, if at a stage 820, the controller 711 determines that the pleth is not of sufficient quality to generate a reliable pulse rate, the controller 711 may return a default value for pulse rate, such as a zero or nonzero pulse rate.

In some examples, further analysis of input data may be part of decision logic 808. In the illustrated example of FIG. 18, decision logic 808 includes a motion analysis 822. The motion analysis 822 may include one or more processes for determining motion features from motion information. The motion analysis 822 may assess one or more features or parameters associated with the motion information. For example, a controller, as part of the motion analysis 822, may assess an amount of motion, determine a type of motion, determine a motion rate, determine a start or stop time of motion, the like or a combination thereof.

In some examples, a controller 711 may assign a motion level or other quantifier to a motion feature in assessing motion in a motion analysis block 822. In some examples, a motion level may be based on a magnitude of one or more peaks in a motion signal (such as one or more of a gyroscope and/or accelerometer signal), or other features in input data determined to be associated with motion and/or motion magnitude. In some examples, the motion level may be a value on a scale associated with different amounts of detected motion. The scale may or may not be a discrete scale. In some examples, the scale may be quantized into stages. For example, a scale may include four stages, where a first stage is associated with no motion and where a fourth stage is associated with strong motion where a pulse is overwhelmed in the pleth signal. A controller 711 may assign a value on this scale to signify the amount of motion identified.

In some examples, a controller, as part of a motion analysis 822, may categorize a type of motion to include random motion, periodic motion, quasiperiodic, or other motion type. In some examples, as part of a motion analysis 822, a controller 711 may identify a motion event. An event identification may include categorization and segmentation of a motion type and determination of a duration of an event based on motion features. For example, a controller 711 may categorize an event, such as walking, running, cycling, swimming, other periodic event or other non-periodic event based on the motion type and duration and/or other motion features. In some examples, a controller 711 may determine an elapsed time during an event after a significant motion event or significant motion feature. For example, running motion may be associated with a significant increase in periodic motion. A controller 711 may determine a running event has occurred if the periodic motion persists after 10, 15, 20, 30 seconds or more or less time has elapsed since the periodic motion started. A combination of gyroscopic and accelerometer signals can give a more powerful way to recognize type and amount of movement. Advantageously, the use of both the gyroscope and accelerometer signals may improve motion type determination over use of just the gyroscope or accelerometer signals because certain types of motion are more dominant in gyroscopic signals and certain types of motion are more dominant in accelerometer signals. For example, gyroscope signals are useful for detecting swing motion due to being sensitive to rotation, such as an arm rotation occurring during walking. In another example, accelerometer signals are useful for detecting step motion, such as footsteps during running. In some examples, the combination of signals may allow for detection of other types of motion, such as rowing, swimming, a swinging motion, and/or others.

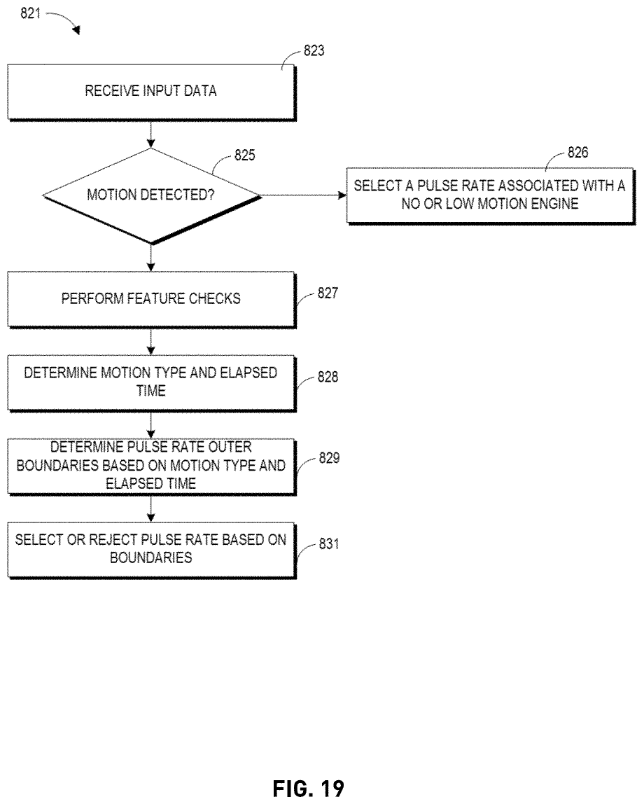

Output of the motion analysis 822, in addition or in the alternative to pleth features, may be used by one or more stages of the decision logic 808 for use in selecting a candidate pulse rate. For example, as illustrated in FIG. 18, a controller 711 may determine, at a decision logic block 824, a candidate pulse rate based on an analysis of the one or more pulse rate candidates, pleth data, and motion features. In some examples, a controller 711 may perform the analysis in multiple steps. For example, in a first step, a controller 711 may determine if and for how long motion is happening. If there is motion, the controller 711 may move on to a second step to determine an appropriate pulse rate for the motion type. If there is not motion, the controller 711 may select a pulse rate calculated or determined using a pulse rate engine with high confidence during no or little motion. FIG. 19 illustrates process 821 that may be part of an example aspect of a decision logic process, such as a second stage decision logic block 824.

As illustrated in FIG. 19, a decision logic process 821 may include a plurality of steps to detect motion and select or reject a pulse rate based on the presence and type of motion in addition to or in the alternative to feature checks. For example, a controller 711 may receive or obtain input data at a block 823. The data may be obtained or received from a previous aspect of decision logic 808, such as a first stage 820, a motion analysis block 822, or other aspect, from one or more of the pulse rate engines, or other source. In some examples, the input data may include, but is not limited to one or more candidate pulse rates from the one or more pulse rate engines, pleth info, and motion info, such as discussed above.

With continued reference to FIG. 19, at a block 825, a controller 711 may determine whether motion is detected during a sampled period. In some examples, a controller 711 may make the determination based on one or more motion features and/or pleth information. For example, a low correlation across two or more channels may indicate the presence of motion. In another example, an amount of motion as determined in a motion analysis may be used to determine presence of motion. For example, a motion analysis 822 may determine a value or score associated with an amount of motion. If the controller 711 determines that a sampled pleth has a no motion score or a score below a threshold level of motion, the controller, may determine that there is no motion. In some examples, a controller 711 may determine the presence or lack of motion based on a combination of checks and/or variables. In some examples, if the controller 711 determines that there is no motion or below a threshold level of motion, at a block 826, may select or reject a pulse rate using a no motion or low motion engine, such as described in U.S. Pat. No. 5,632,272, titled “SIGNAL PROCESSING APPARATUS,” issued May 27, 1997, U.S. Pat. No. 6,002,952, titled “SIGNAL PROCESSING APPARATUS AND METHOD,” issued Dec. 14, 1999, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,463,311, titled ‘PLETHYSMOGRAPH PULSE RECOGNITION PROCESSOR,” issued Oct. 8, 2002, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein.

If the controller 711 determines that a sampled pleth has motion or exceeds a threshold level of motion, the controller 711 may move on to block 827.

At a block 827, a controller 711 may perform one or more feature checks on the input data. For example, a controller 711 may select, average, or hold pulse rate values based on one or more time domain feature checks. In some examples, one or more features checks may include one or more checks based on motion type. In some examples, a controller 711 may perform flexible (or adaptive) smoothing on pleth data based on a trend history. In some examples, a controller 711 may average final pulse rate based on a history of pulse rate values or reject the final pulse rate.

At a block 828, a controller 711 may determine a motion type and elapsed time. In some examples, the motion type and elapsed time may be determined at a motion analysis block 822. As referenced above, a motion type may include random motion, periodic motion, quasiperiodic, or other motion type. In some examples, a motion type can include a more specific categorization, including, but not limited to walking, running, cycling, swimming, other periodic event or other non-periodic event based on the motion type and duration and/or other motion features. In some examples, a controller 711 may determine an elapsed time during an event after a significant motion event or significant motion feature.

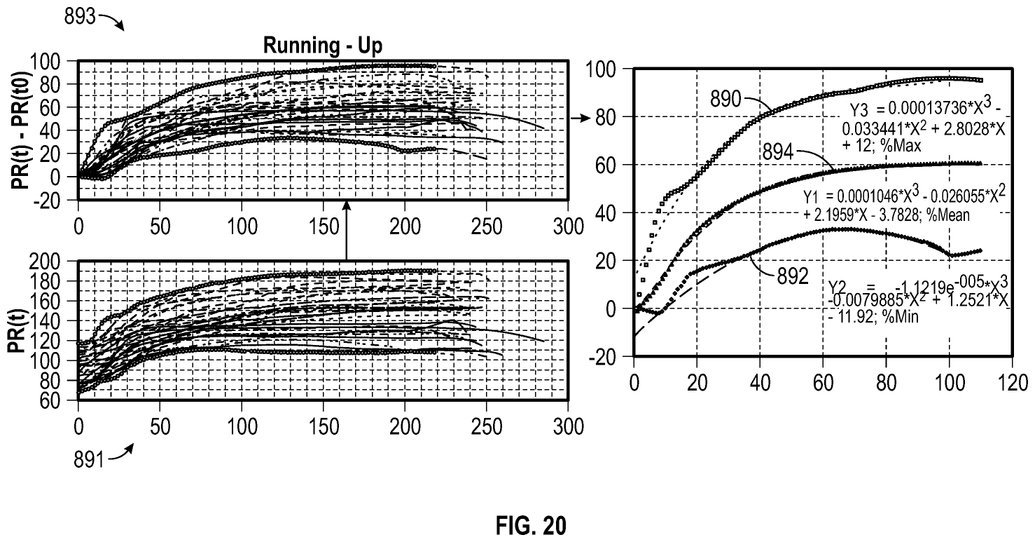

At a block 829, a controller 711 may determine an upper and/or lower boundary for a pulse rate based on the motion type and/or elapsed time. The upper and/or lower boundary may be based on empirical data for average heart rates or changes in heart rate over a similar elapsed amount of time as has elapsed for a user of the device since a motion event began and during a similar event type as the user is engaging in. For example, a set of boundaries may be dependent on elapsed time of a motion type and be different based on the motion type. For example, a set of boundaries may be different for running, swimming, walking, and cycling. In some examples, the set of boundaries may be dependent on other variables, such as a detected amount of motion, physical fitness of a user, or other parameter. FIG. 20 illustrates example empirical data across a range of users over illustrates a change in heart rate over a period of approximately 250 minutes for a running motion type. As illustrated in graph 891, different users may have different resting heart rates or starting heart rates. In order to account for the variation in resting or starting heart rate, the empirical data may be normalized, such as shown in graph 893, so that a starting value is zero. Using the normalized data, an upper boundary 890, lower boundary 892, and average shape 894 for a pulse rate may be calculated that is agnostic of starting heart rate. These boundaries may then be used by the controller 711 to select or reject a candidate pulse rate.

With continued reference to FIG. 19, at a block 831, a controller 711 may select or reject a pulse rate determined or calculated by the one or more pulse rate engines based on whether the pulse rate falls within or outside the boundaries determined at block 829. For example, if a candidate pulse rate falls within the boundaries, then a controller 711 may select or not reject the candidate pulse rate. However, if a candidate pulse rate falls outside the boundaries, then a controller 711 may reject or not select the candidate pulse rate. In some examples, this process may select a candidate pulse rate as the candidate pulse rate to output as the pulse rate. In some examples, this process may result in a plurality of candidate pulse rates and further decision logic may be used to determine an output pulse rate.

### Example Blood Oxygen Saturation Determination

A controller 711 may determine a saturation value (SpO2 value) using some combination of PPG signals, including an infrared signal, red signal, and a green signal. In some examples, a red signal may have a wavelength at approximately 620 nm or 660 nm. Advantageously, the use of 620 nm over 660 nm may improve a signal quality due to stronger absorption at a measured tissue site. It is of note that while the signal strength is improved, hemoglobin absorption's wavelength dependence at 620 is steeper than at 660, which may need to be compensated for in processing. In some examples, an infrared signal have a wavelength at approximately 970 nm (or IR970) in addition or in the alternative to the standard infrared signal may be used to help calibrate the signals and accordingly produce a more accurate saturation determination. For example, since IR970 is sensitive to fluctuations in user hydration, it can be used to account for hydration changes that may affect changes in the signals. As the wavelength dependent absorption coefficient of (de)oxyhemoglobin varies significantly across the emission spectrum of the 620 nm LED, the amount of interrogated blood can bias the emission spectral distribution of diffusely reflected 620 nm light. Therefore as the SpO2 calibration curve of 620/905 nm LEDs is somewhat sensitive to amount of blood, IR970 may be used to correct for residual SpO2 error based on the content of water within the blood. It is of note that and IR970 can also be used by the controller to generate a hydration index using a hydration engine 720. When used in conjunction with a normalizing light source(s) (e.g. {525, 620, 660, 905, etc} nm LEDs), there is a specific absorption peak of water around 970 nm that may attenuate 1) the absolute intensity of 970 nm light to estimate overall tissue hydration, and 2) the pulsatile intensity of 970 nm light to estimate the aqueous content of the blood.

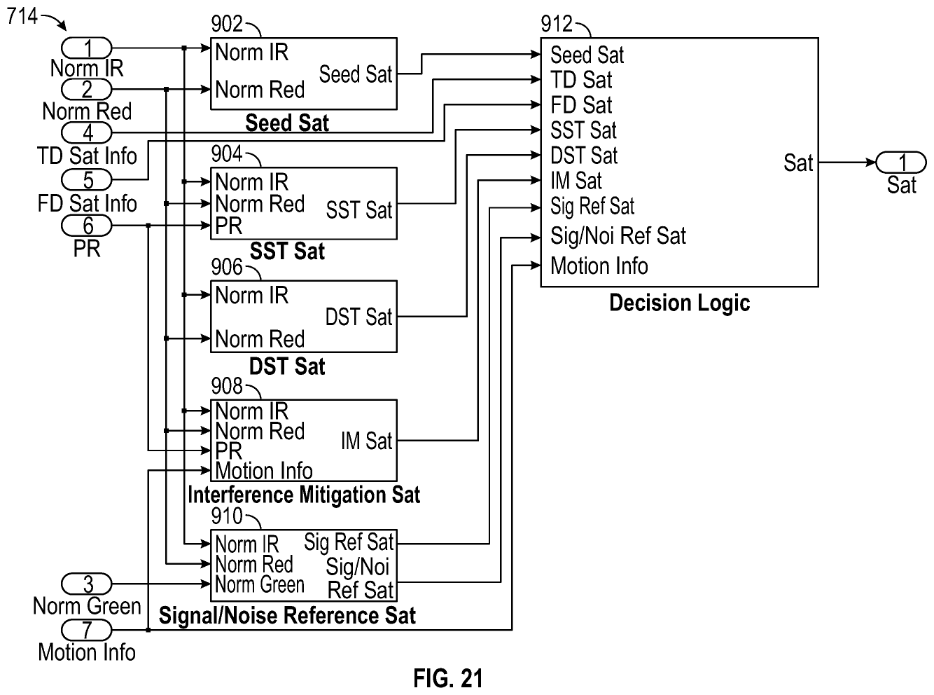

In some examples, an SpO2 determination engine 714 may include a plurality of individual SpO2 determination engines. FIG. 21 illustrates an example SpO2 determination engine 714. For example, an SpO2 determination engine 714 may include, but is not limited to, a seed saturation engine 902, an SST saturation engine 904, a DST saturation engine 906, an interference mitigation saturation engine 908, a noise reference saturation engine 910, a frequency domain saturation engine (not shown), or other engine. One or more aspects of engines, including a seed saturation engine 902, SST engine 904, DST engine 906, FST engine, MST engine, and/or other engines such as described in U.S. Pat. No. 5,632,272, titled “SIGNAL PROCESSING APPARATUS,” issued May 27, 1997, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein.

In some examples, calculated values from one or more of the engines may be output or bundled for further processing. For example, a seed saturation engine 902 may be configured to output a seed saturation value, an SST saturation engine 904 may be configured to output an SST saturation value, a DST saturation engine 906 may be configured to output a DST saturation value, an interference mitigation saturation engine 908 may be configured to output an interference mitigation (or IM) saturation value, and a noise reference saturation engine 910 may be configured to output a signal reference saturation value and/or a signal/noise reference saturation value. In addition or in the alternative to the above values, a time domain saturation value or saturation information and/or a frequency domain saturation value or saturation information may be analyzed or output by an SpO2 (or saturation) determination engine 714. In some examples, the time domain saturation information or value and/or the frequency domain saturation information or value may be determined at least in part by a pulse rate determination engine 718. In some examples, motion information may also be analyzed or output by the saturation determination engine 714. In some examples, the motion information may be determined at least in part by a pulse rate determination engine 718.

A seed saturation engine 902 may include at least one process or algorithm for determining an SpO2 value using a combination of PPG signals, such as a normalized IR signal and a normalized red signal. In some examples, an SpO2 signal may be determined based on a ratio of PPG signals, such as a ratio of a Red and IR channels. An SST saturation engine 904 may include at least one process or algorithm for determining an SpO2 value using a combination of PPG signals, such as a normalized IR signal and a normalized Red signal, and pulse rate, such as determined using a pulse rate engine 718. A DST saturation engine 906 may include at least one process or for determining an SpO2 value using a combination of PPG signals, such as a normalized IR signal and a normalized red signal. An interference mitigation saturation engine 908 may include at least one process or for determining an SpO2 value using a combination of PPG signals, pulse rate, and motion information. In some examples, the PPG signals may include a normalized IR signal, a normalized Red signal, and a normalized Green signal. In some examples, the pulse rate and/or motion information may be determined based on an output of the pulse rate engine 718. A noise reference saturation engine 910 may include at least one process or for determining an SpO2 value using a combination of PPG signals, such as a normalized IR signal, normalized Red signal, and normalized Green signal.

An interference mitigation engine 908 may use some combination of PPG signals, pulse rate, and motion information to generate a saturation value and/or a corresponding confidence score. In some examples, a controller 711 may use a plurality of sub-engines to determine a saturation value.

In some examples, an interference mitigation engine 908 may use pulse rate from a pulse rate engine 718 to select a correct peak in the PPG spectrum in order to generate a red/IR ratio that may be used to determine a saturation value. For example, a controller 711 may determine if a pulse estimate is accurate or reliable. In some examples, this determination may be done by decision logic in a pulse rate determination engine 718 and/or may be based on one or more checks, such as whether detected motion is near the pulse rate and whether harmonics are detected for the selected pulse rate. If the pulse rate is sufficiently accurate, then the controller 711 may use the location of the pulse rate peak in the frequency domain to estimate a location of an accurate portion of PPG signal so that an accurate red/IR ratio may be determined that can be used to estimate a saturation value.

In some examples, an interference mitigation engine 908 may include one or more processes for cleaning up a signal, such as a Green PPG signal, Red PPG signal, or IR PPG signal For example, a controller 711 may generate or use a noise reference based on some combination of gyroscope axis signals, some combination of accelerometer axis signals, an integral of at least one accelerometer axis (which may correspond to a velocity value), a double integral of at least one accelerometer axis (which may correspond to a position value), an integral of at least one gyroscope axis, an infrared PPG signal, a red PPG signal, some combination of gyroscope axis and accelerometer axis signals as multiple noise references, Green PPG signal, the like or a combination thereof. In some examples, a controller 711 may use motion information, such as some combination of gyroscope and accelerometer signals to remove or reduce noise in one or more of the PPG signals, such as a red signal and/or IR signal. In some examples, a green PPG signal may be used as a signal reference and a controller 711 may project a red PPG signal and IR PPG signal on to a green PPG signal subspace to generate a noise reference for cleaning up a red PPG and/or IR PPG signal for determination of a saturation value. In some examples, a green PPG signal may be used as a signal reference for a least mean squares (LMS) algorithm, where a red PPG and IR PPG signal are used as a noise reference. In some examples, noise cancellation may be done in FFT space.

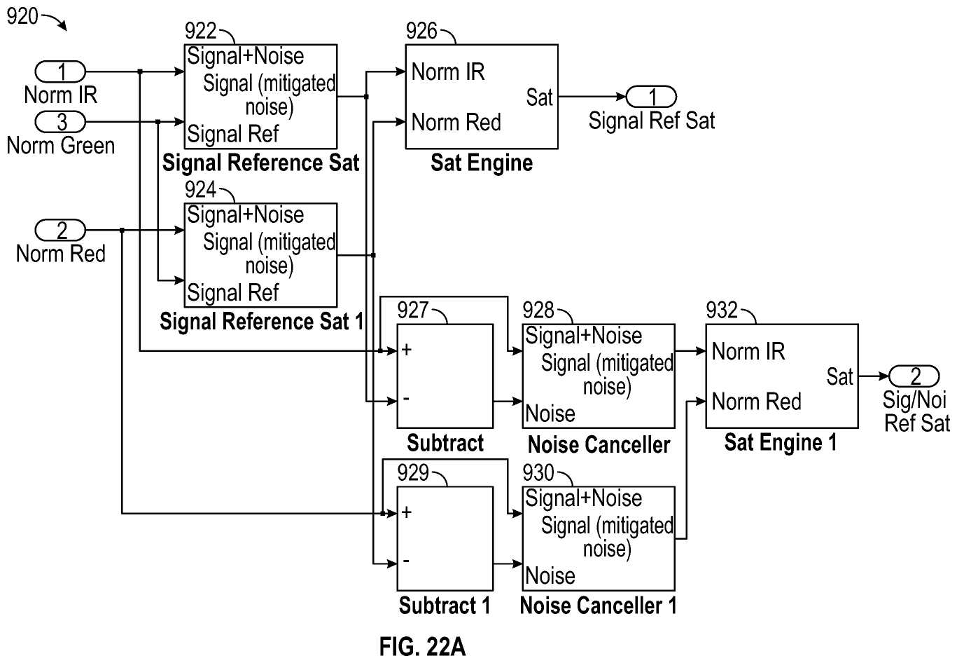

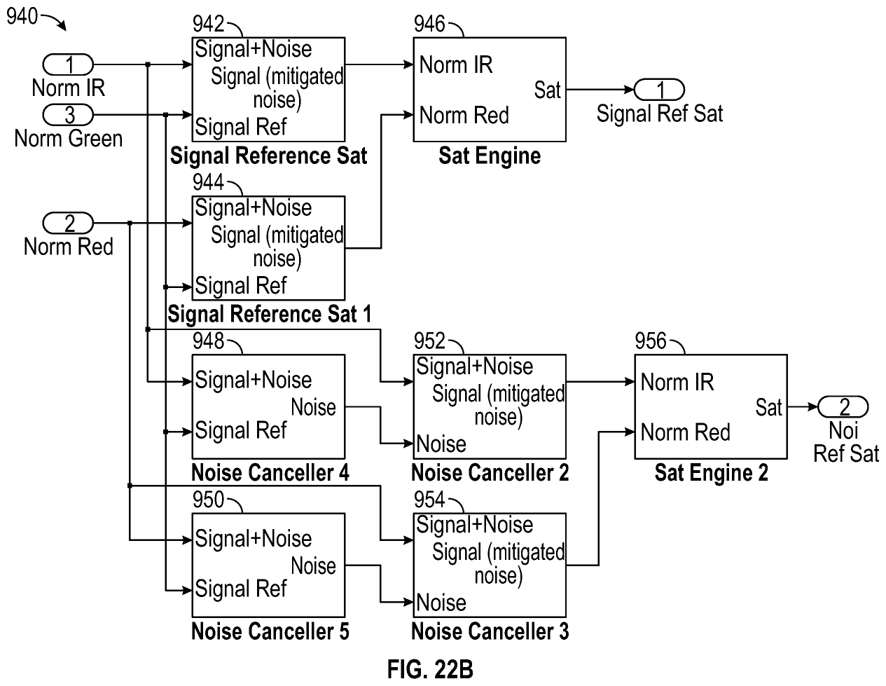

A noise reference saturation engine 910 may use one or more processes for cleaning up a spectrum in order to generate a saturation value. FIG. 22A and FIG. 22B illustrate example processes 920, 940 that may be used by a noise reference saturation engine 910 to determine a signal reference saturation estimation and/or a noise/signal reference saturation estimation.

In some examples, a controller 711 may use a green PPG signal as a signal reference. For example, a controller 711 may project red PPG and/or IR PPG signals on the green PPG signal subspace and subtract that projection from red PPG and/or IR PPG signal to determine a noise reference. The controller 711 may use noise cancellation to remove the noise reference from the red PPG/IR PPG. The resulting red PPG and/or IR PPG can then be used to estimate saturation.

In some examples, such as illustrated in FIG. 22A, a controller 711 may receive a green signal as a signal reference and an IR signal as a signal plus noise reference and generate an IR signal with mitigated noise at a block 922. A controller 711 may receive a green signal as a signal reference and a red signal as a signal plus noise reference and generated a red signal with mitigated noise at a block 924. The mitigated red and IR signals may be processed at a block 926 to determine a signal reference saturation estimate.

With continued reference to FIG. 22A, In addition or in the alternative to the signal reference saturation estimate, a controller 711 may determine a signal/noise reference saturation estimate using the mitigated red and IR signals. For example, a controller 711 at block 927 may subtract a mitigated IR signal from an input IR signal to generate an IR noise reference. The controller 711 may use a noise canceller at a block 928 to remove the IR noise reference from the IR input signal to generate an IR signal with mitigated noise. Additionally, a controller 711 at block 929 may subtract a mitigated Red signal form an input Red signal to generate a red noise reference. The controller 711 may use a noise canceller at a block 930 to remove the red noise reference from the Red input signal to generate a red signal with mitigated noise. The IR signal with mitigated noise and the red signal with mitigated noise may be processed at a block 932 to determine a signal/noise reference saturation estimate.

In some examples, a controller 711 may use the green PPG as a ‘noise’ reference. A controller 711 may use noise cancellation to remove the ‘noise’ reference. For example, a controller 711 may treat Red and/or IR PPG signals as signal plus noise and green PPG as signal. Accordingly, noise cancellation will remove the signal from the red PPG and/or IR PPG and output the noise. The output of the noise canceller may act as a noise reference to another noise canceller with Red/IR PPG as signal plus noise.

In some examples, such as illustrated in FIG. 22B, a controller 711 may receive a green signal as a signal reference and an IR signal as a signal plus noise reference and generate an IR signal with mitigated noise at a block 942. A controller 711 may receive a green signal as a signal reference and a red signal as a signal plus noise reference and generate a red signal with mitigated noise at a block 944. The mitigated red and IR signals may be processed at a block 946 to determine a signal reference saturation estimate.

With continued reference to FIG. 22B, In addition or in the alternative to the signal reference saturation estimate, a controller 711 may determine a signal/noise reference saturation estimate using the input IR and red signals as signal plus noise and the green signal and a signal reference. For example, a controller 711 at block 948 may use a noise canceller at a block 948 to generate an IR noise reference using a green signal as a signal reference and an IR input signal as signal plus noise. In some examples, a controller 711 may use another noise canceller at a block 952 to generate an IR signal with mitigated noise using the IR noise reference from block 948 and the input IR signal as signal plus noise. A controller 711 at a block 950 may use a noise canceller at a block 950 to generate a red noise reference using a green signal as a signal reference and a red input signal as signal plus noise. In some examples, a controller 711 may use another noise canceller at a block 954 to generate a red signal with mitigated noise using the red noise reference from block 950 and the input red signal as signal plus noise. A controller 711 may process the mitigated red and IR signals at a block 956 to generate a noise/signal saturation estimate.

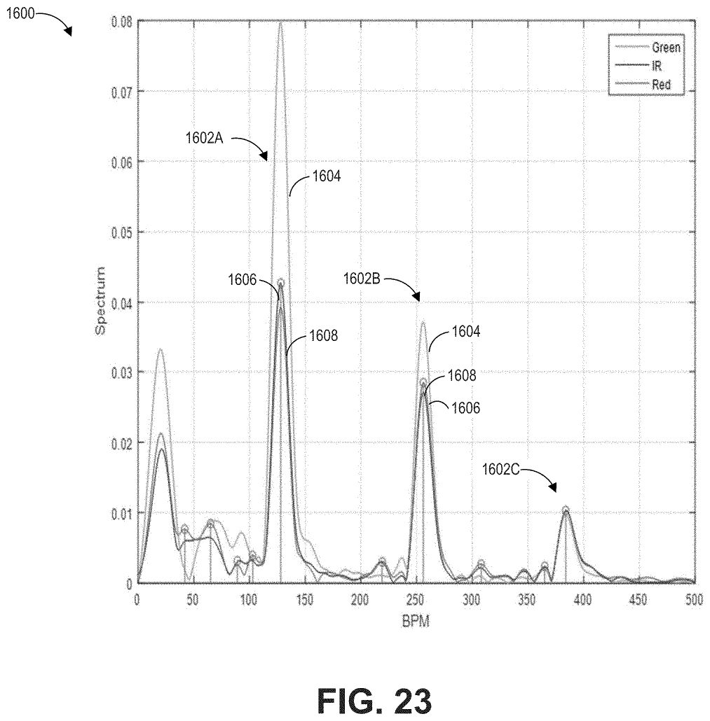

In some examples, a controller 711 may determine a saturation estimate using a frequency domain (FD) saturation engine. FIG. 23 illustrates example empirical data 1600 that may be analyzed by a controller using a frequency domain saturation engine. A FD saturation engine may analyze peaks in the frequency domain of pleth signals, including but not limited to a green signal 1604, red signal 1606, and IR signal 1608, to determine a saturation estimate. For example, a controller 711 may determine one or more candidate peaks associated with a pulse or other physiological process according to one or more candidate peak selection processes. A candidate peak may, for example, have harmonics associated with peaks at other frequencies in the pleth signal. The controller 711 may analyze the candidate peak(s) and/or its harmonics to determine one or more ratios of signals at each peak and weight those ratios to determine a saturation estimate. The number of candidate peaks 1602A, 1602B, 1602C analyzed may be one, two, three, or more candidate peaks. In some examples, the controller 711 may analyze three candidate peaks.

With continued reference to FIG. 23, each peak 1602A, 1602B, 1602C may include a peak in green signal 1604, red signal 1606, and IR signal 1608. The green signal 1604 may have a stronger signal at the candidate peak 1602A, 1602B, 1602C than the red signal 1606 and IR signal 1608. The controller 711 may determine a ratio of a red signal 1606 over an IR signal 1608 at each candidate peak and weight each calculated ratio based on the magnitude of the green signal 1604. The controller 711 may apply a greater weight to a stronger green signal at a peak than a weaker green signal. The controller 711 may then calculate a mean or perform another calculation to combine the or select the weighted ratios across the analyzed candidate peaks 1602A, 1602B, 1602C. The controller 711 may output a candidate saturation estimate based on the calculation. For example, the controller 711 may output the weighted mean of candidate peaks as the saturation estimate for the frequency domain saturation engine. Advantageously, weighting the calculated ratios by a green signal 1604 may improve a saturation estimate because ratios at different peaks may be skewed by artifacts in the red or IR signal, such as due to motion. The green signal is stronger and less likely to be skewed due to motion. Thus, weighing the ratios by the stronger signal may result in a more accurate saturation estimate than without the use of the stronger, green signal.

Decision logic 912 may be configured to receive one or more inputs from the one or more saturation engines, other engines and/or other sources. Inputs may include, but are not limited to a seed saturation value, a time domain saturation value, a frequency domain saturation value, an SST saturation value, a DST saturation value, an interference mitigation saturation value, a signal reference saturation value, a signal/noise reference saturation value, motion information, a frequency domain saturation value, and confidence values associated with any of the individual saturation values determined by the engines. The decision logic 912 may include one or more post-processing steps, such as averaging or smoothing steps. In some examples, the decision logic 912 may be configured to select or reject saturation values in order to determine a saturation value to output. The selected saturation value by the decision logic 912 (also sometimes referred to as raw saturation) and/or series of raw saturation values may be weighted and averaged together as a smoothed output. In some examples, a controller 711 may average, filter, or otherwise process saturation estimations before sending to decision logic 912. In some examples, each engine can be configured to buffer up past estimates and average them before passing to the decision logic 912.

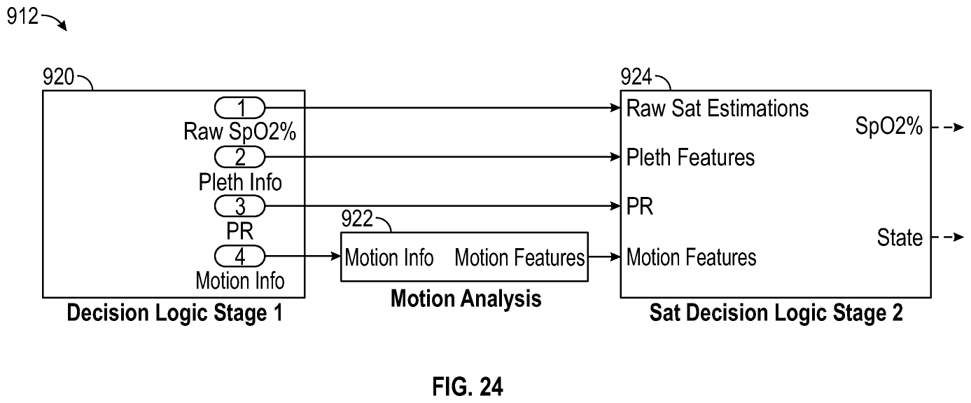

FIG. 24 illustrates aspects of decision logic 912 that may be used to select a candidate saturation from one or more of the candidate saturation estimates determined by the one or more saturation engines, such as engines 902, 904, 906, 908, and 910. For example, decision logic 912 may include a plurality of stages, such as a first stage 920 and a second stage 924. It is of note that while a first stage 920 and a second stage 924 are illustrated as performed in a certain order, the decision logic stages may be performed in any suitable order. In some examples, a controller 711 may process data before, after, or between decision logic stages. For example, as illustrated in FIG. 24, a controller 711 may perform a motion analysis 922 of motion information determined by the controller 711 at an earlier stage.

One or more inputs to the decision logic 910 or aspects of the decision logic 910 may include one or more candidate saturation estimates from the one or saturation engines, pleth info, pulse rate, and motion info. In some examples, the one or more candidate saturation estimates may include one or more data types, including but not limited to the calculated saturation estimates from the individual engines and one or more associated confidence scores associated with the calculated saturation estimates. In some examples, pleth info may include one or more data types. For example, pleth info may include some or all of the same or similarly determined pleth info such as described above with reference to the pulse rate engine 718. For example, pleth info may include time domain and frequency domain features for some or all of the channels of the pleth signal. In some examples, the number of channels may include 4, however more or fewer channels are also possible. In some examples, the pleth info may include pleth features related to saturation, including but not limited to DC features for all channels and/or a DC ratio.

A DC signal associated with a channel, such as a red channel or signal and an IR channel or signal, may include a pleth channel with a removed AC component. In some examples, a controller 711 may remove an AC component using a low pass filter or by taking the mean of the frame snapshot. A controller 711 may generate a DCTrend for use in selecting or rejecting a saturation estimate. Advantageously, a DCTrend may contain information related to a saturation trend, such as whether a saturation estimate is rising or falling. If a saturation estimate is counter to the saturation trend, a controller 711 may use this information to reject the saturation estimate as the selected candidate saturation estimate. Advantageously, use of DC may be helpful in the saturation estimate when used on a wrist over a finger because a finger. For example, the DC value can indicate if SpO2 is changing. If the DC value hold steady and there is a lot of motion, the DC value can tell you that the previous SpO2 values are holding steady, which can inform the decision logic in selecting a measurement to output. Similarly, if the DC value is going up or down, you can take that information and make assumptions about where an accurate SpO2 value can be found. Advantageously, using DC values may help improve pleth measurements when taken from the wrist or other complex tissue. SpO2 measurements at the wrist rely on diffuse reflectance of complex tissue, rather than transmission which is used on the more homogenous tissue qualities of the fingertip capillaries. The skin at the wrist consists of shallow capillaries and deeper vessels. As the optically observable perfusion of the capillary bed may be weak compared to deeper venous pulsations (which may be significantly augmented by movement), advantageously, the DC trend may allow for interpolation of SpO2 during motion periods excessive motion. In some examples, a DCTrend may be determined using a DC ratio and a DC baseline. A DC ratio may include a ratio of a DC IR signal and DC red signal:

DCRatio=DC_red/DC_IR

A DC baseline may include a moving average of a DCRatio during no motion. In some examples, a DC baseline can be held or not updated during serious motion. Advantageously, holding the baseline may prevent the DC baseline from being biased by excessive motion. A controller 711 may generate a DCTrend as a function of the DC ratio and DC baseline:

DCTrend=((DCRatio−DCBase)/DCBase)×100(%)

In some examples, motion information may include, but is not limited to, information associated with motion of the user or device. For example, motion information may include, but is not limited to motion features, type of motion (for example, periodic, random), motion rate, magnitude of a gyroscope signal, magnitude of an accelerometer signal, maximum acceleration, or other motion related information. In some examples, motion information may include data output from one or more pulse rate engines. In some examples, motion information may include signal associated with a six degree of freedom inertial measurement unit (IMU).

In some examples, the motion information and analysis may be the same or similarly determined motion information and motion features such as described with reference to the pulse rate engine 718. In some examples, analysis of input data may be part of decision logic 912. Additionally or alternatively, analysis of the input data may be determined by a pulse rate engine 718 and used in the saturation engine 714 and saturation engine decision logic 912. In the illustrated example of FIG. 24, decision logic 912 includes a motion analysis 922. The motion analysis 922 may include one or more processes for determining motion features from motion information. The motion analysis 922 may assess one or more features or parameters associated with the motion information. For example, a controller, as part of the motion analysis 922, may assess an amount of motion, determine a type of motion, determine a motion rate, determine a start or stop time of motion, the like or a combination thereof. Outputs of the motion analysis 922 may be similar to the outputs described above with reference to the motion analysis 822 described with reference to FIG. 18.

One or more outputs of the decision logic 912 may include, but is not limited to, a selected candidate saturation estimate and a state value. The state value may include, for example, a tracking value associated with the selected saturation estimate configured to illustrate the path (or engine) used to determine the selected saturation estimate. In another example, the state value may additionally or alternatively include a confidence measure. In some examples, the state value may indicate the type of logic, rule, and/or features used to generate a decision. This can help explain why a decision is made by the decision logic 912 and the explanation itself can additionally or alternatively be a feature for analysis.

In some examples, a decision logic stage 920 may include a first saturation estimate selection or determination based on one or more selection processes, such as described in U.S. Pat. No. 5,632,272, titled “SIGNAL PROCESSING APPARATUS,” issued May 27, 1997, U.S. Pat. No. 6,606,511, titled “PULSE OXIMETRY PULSE INDICATOR,” issued Aug. 12, 2003, U.S. Pat. No. 6,684,090, titled “PULSE OXIMETRY DATA CONFIDENCE INDICATOR,” issued Aug. 12, 2003, U.S. Pat. No. 6,430,525, titled “VARIABLE MODE AVERAGER,” issued Aug. 6, 2002, U.S. Pat. No. 6,999,904, titled “VARIABLE INDICATION ESTIMATOR,” issued Feb. 14, 2006, U.S. Pat. No. 6,850,787, titled “SIGNAL COMPONENT PROCESSOR, issued Feb. 1, 2005, and U.S. Pat. No. 9,211,095, titled “PHYSIOLOGICAL MEASUREMENT LOGIC ENGINE,” issued Dec. 15, 2015, the entire contents of each of which are incorporated by reference herein. In some examples, the decision logic stage 920 may serve to select an initial saturation estimate.

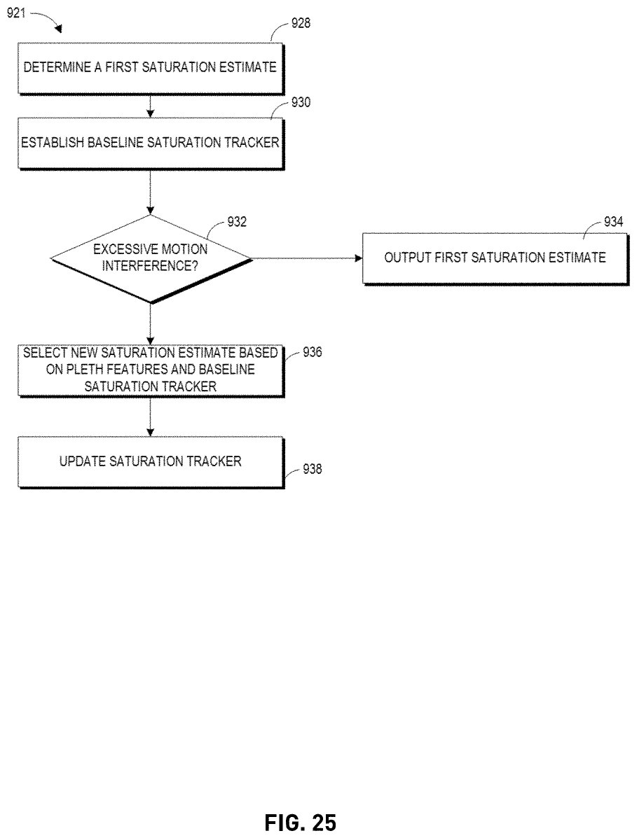

An output of the decision logic 920 may be maintained as the output of the saturation determination engine 714 or rejected by a second decision logic stage 924. FIG. 25 illustrates an example process 921 that may be part of a decision logic stage 924. In some examples, a decision logic process 921 may have a plurality of steps or stages. For example, a decision logic process 921 may include a first saturation estimate block 928, baseline establishing block 930, a motion interference check block 932, a first saturation selection block 934, a second saturation selection block 936, a baseline updating block 938, or more, fewer, or different blocks.

In a first saturation estimate block 928, a controller 711 may determine a first saturation estimate. In some examples, the first saturation estimate may be the output of the first decision logic stage 920. In some examples, the first saturation estimate may be an estimate calculated by one or more saturation engines more accurate under no motion or low motion. For example, the first saturation estimate may be calculated by a seed saturation engine, SST engine, DST engine, or the like.

In a baseline establishing block 930, a controller 711 may determine or calculate a saturation tracker baseline. In some examples, a saturation tracker baseline may be based on a DCTrend baseline. As described above, a DCTrend may be a function of a DCRatio and DC base:

DCTrend=((DCRatio−DCBase)/DCBase)×100(%)

Where a DCRatio is a ratio of a DC component of a red signal and a DC component of an IR signal and a DCBase is a moving average of a DCRatio during no motion. A saturation trend may be determined based on the DCTrend. For example, if a DCTrend is increasing, a saturation may correspondingly increase and if a DCTrend is decreasing, may correspondingly decrease.

In a motion interference check block 932, a controller 711 may determine whether there is excessive motion interference in the one or more PPG signals. The controller 711 may determine whether there is motion interference using at least one motion interference check. An interference check may include, but is not limited to, a determination whether there is too much variation in a saturation trend, a cross correlation check, a motion check based on one or more motion features, and a motion rate and pulse rate similarity check. If a controller 711 determines there is no excessive motion interference, the controller 711 may select or output the first saturation value determined at block 728.

In some examples, a variation in a saturation trend may be determined based on a slope of a saturation trend, a direction of a determined trend, the like or a combination thereof. For example, if a variation in a saturation trend is too high, a controller 711 may determine that there is excessive motion interference. In another example, if a direction of a trend is in a different direction than expected based on other indicators, the controller 711 may determine that there is excessive motion interference.

In some examples, a cross correlation check may include a check of the correlation of channels in the input signals. For example, a cross correlation may include a correlation of red and IR signals, a correlation of green and red signals, a correlation of green and red signals, the like or a combination thereof. If a controller 711 determines a poor correlation across channels, then the controller 711 may determine that there is excessive motion interference. In some examples, a controller 711 may determine that there is excessive motion interference if there is poor correlation in two or more channels. A quality of correlation may be based on a correlation score, such as a value between 0 and 1, where 0 corresponds to no correlation and 1 corresponds with excellent correlation. In some examples, a controller 711 may determine that there is poor correlation where the correlation score falls below a threshold value. If the controller 711 determines that there is poor correlation, the controller 711 may determine that there is excessive motion interference.

In some examples, a motion check may include a determination that there is greater than a threshold level of motion based on the one or more motion features, such as described above. For example, a controller 711 may determine a motion level or score associated with a pleth based on one or more motion features. If the motion level or score is greater than a threshold, then the controller 711 may determine that there is excessive motion interference. Other determinations of motion based on the one or more motion features may also be possible.

In some examples, a pulse similarity check may include a determination that a pulse rate is close to a rate of motion. For example, if a pulse rate determined by a pulse rate engine 718 is within a threshold distance on a spectrum from a rate of motion, the controller 711 may determine that there is excessive motion interference. For example, if a pulse rate is within 10 beats per minute, 5 beats per minute, 1 beat per minute, or more or less beats per minute from a determine rate of motion, the controller 711 may determine that there is excessive motion interference.

If a controller determines that there is excessive motion interference, a controller 711 may select or reject a new saturation value at a block 936. The new saturation value may be a saturation value determined by a saturation engine that is more likely to be accurate during motion or excessive movement. For example, the new saturation value may be determined by an interference mitigation saturation engine 908 or a signal/noise reference saturation engine 910. In some examples, the controller 711 may select a new saturation value based on a confidence value associated with a calculated saturation value from an engine. Additionally or alternatively, a controller 711 may select a new saturation value based on one or more pleth features, match between saturation trend based on the DCTrend and calculated trend from a saturation engine, motion features, or other parameters. For example, a frequency domain (FD) saturation value may be selected if motion is observed, PR and motion rates are not similar and the change rate between a FD saturation value and the baseline saturation tracker generate the best match with a DCTrend.

In a baseline updating block 939, a controller 711 may update a saturation trend by calculating, for example an updated DCTrend. The updated saturation trend may be used to determine a new saturation value at a future time. The updated saturation trend may be updated using the selected saturation value.

In some examples, the decision logic process 912 may select a candidate saturation value to output as the saturation value. In some examples, this process may result in a plurality of candidate saturation values and further decision logic may be used to determine an output saturation value.

### Example Motion Data Processing

In some examples, a controller 711 may pre-process one or more motion signals, such as a gyroscope and/or accelerometer signal or signals as part of a pre-processing engine 712. Advantageously, a controller 711 may use a pleth clock to generate a higher precision gyroscope and/or accelerometer data at a desired sample rate without introducing excessive noise in the data.

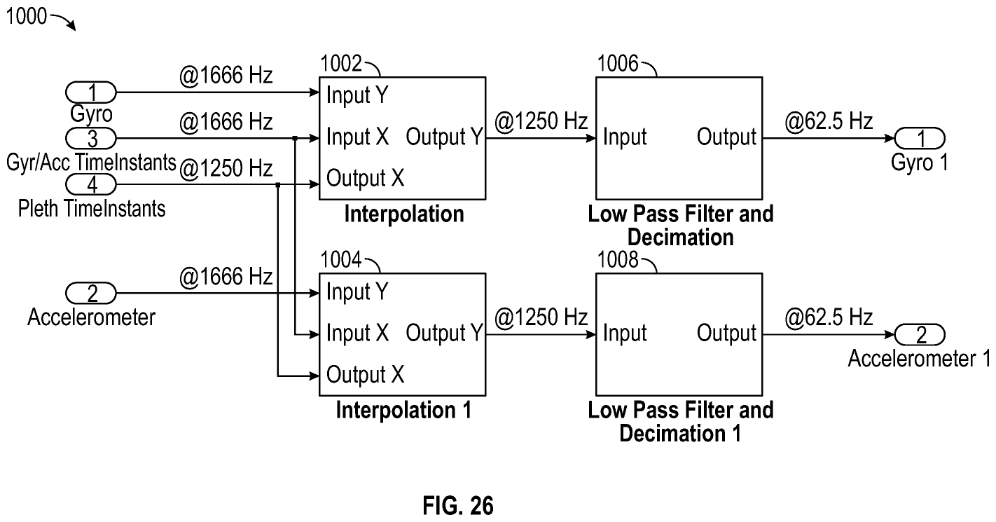

FIG. 26 illustrates an example motion processing algorithm 1000 that may be part of a pre-processing engine 712. The process may include a first interpolation block 1002, a second interpolation block 1004, a first filter and decimation block 1006, and second filter and decimation block 1008 or more or fewer blocks. In some examples, a controller 711 may process one or more gyroscope signals, gyroscope and/or accelerometer time instants, and pleth time instants at a block 1002 to interpolate a value of one or more gyroscope signals at a desired sample rate that may align the pleth instants. The sample rate can include, for example, 1250 Hz, or more or fewer Hz. In some examples, a controller 711 may filter and/or decimate the interpolated signal at a block 1006 to generate at least one gyroscope signal at a reduced frequency that may be used by the controller 711 in analysis of or using the gyroscope signal. The reduced sample rate can include, for example, 62.5 Hz, or more or fewer Hz. In some example, a controller 711 may process one or more accelerometer signals, gyroscope and/or accelerometer time instants, and pleth time instants at a block 1004 to interpolate a value of one or more accelerometer signals at a desired sample rate that may align the pleth instants. The sample rate can include, for example, 1250 Hz, or more or fewer Hz. In some examples, a controller 711 may filter and/or decimate the interpolated signal at a block 1008 to generate at least one accelerometer signal at a reduced frequency that may be used by the controller 711 in analysis of or using the accelerometer signal. The reduced sample rate can include, for example, 62.5 Hz, or more or fewer Hz.

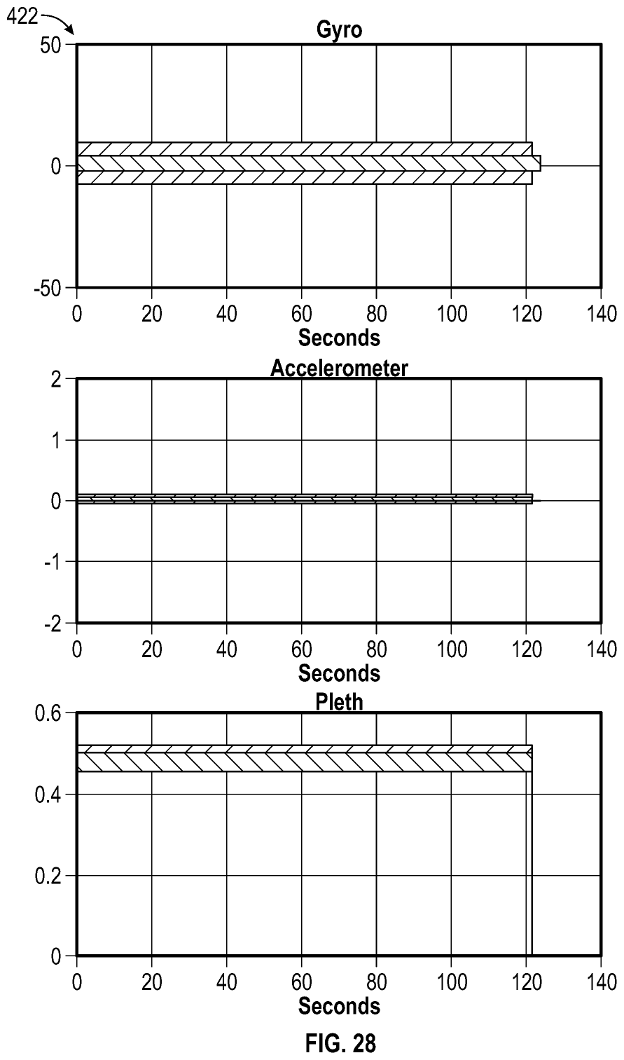

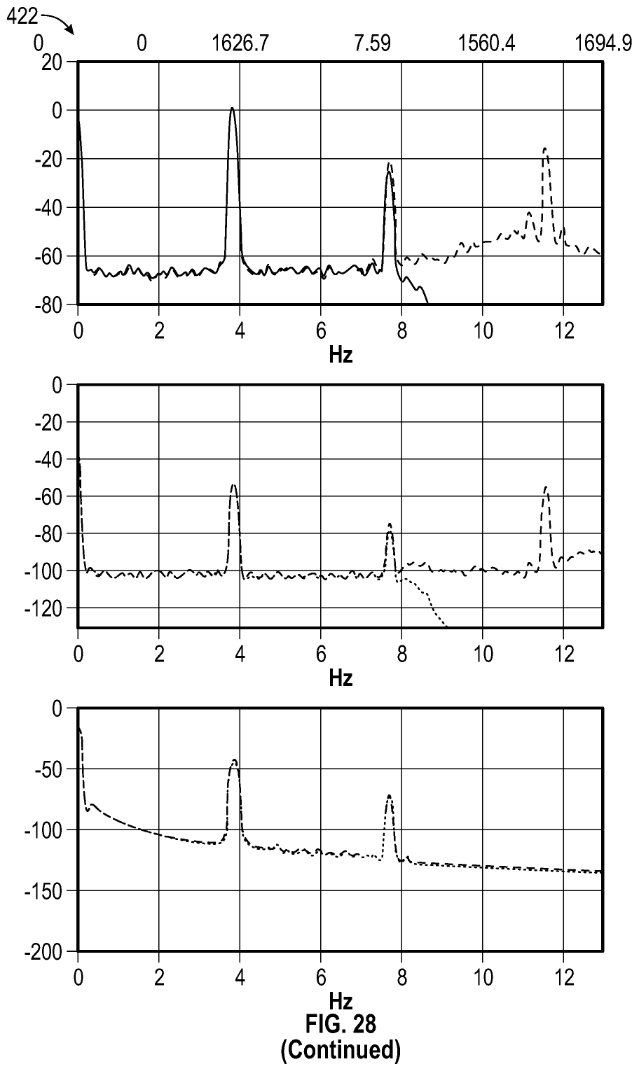

FIG. 27 and FIG. 28 illustrate example gyroscope and accelerometer signals without and with processing to generate signals at a desired sample rate.

### Example Error Reduction Features

As discussed above, the plethysmograph module 100, 300 can determine a wearer's SpO2. For traditional pulse oximeters, there may be gaps in the SpO2 measurement for certain lengths of time due to, for example, movement of the wearer (e.g., the wearer's finger, wrist, or other body part). These gaps can cause low confidence levels in the SpO2 measurement. As further described below, the plethysmograph module 100, 300 can bridge these gaps in the SpO2 determination when the confidence levels are low by using a dynamic calibration curved based on the ratio of DC components of detected signals generated in response to light corresponding to different wavelengths interacting with a tissue site of the wearer. Advantageously, the ratio of DC components is less effected by a wearer's movement.

As discussed above, the emitters 104, 304a, 304b of the plethysmograph module 100, 300 can emit light corresponding to multiple wavelengths, such as a second wavelength and a third wavelength. In particular, the second wavelength can be about 620 nm and the third wavelength can be about 905 nm. The device processor and/or plethysmograph module processor can determine a ratio of DC components of detected signals generated in response to the light corresponding to the second and third wavelengths interacting with the tissue site of the wearer. The device processor and/or plethysmograph module processor can store the calculated ratio of DC components over a period of time when the confidence level of the SpO₂ determination is high (e.g., when the wearer is not moving). For example, the wearer's SpO₂ can be estimated based on the time-domain pulse rate (TDPR) and the frequency-domain pulse rate (FDPR). If the SpO₂ estimations based on the TDPR and FDPR are similar or the same, the confidence level may be high. Alternatively, if the SpO2 estimations based on the TDPR and FDPR are different, the confidence level may be low.

The device processor and/or plethysmograph module processor can average the ratio of DC components over the period of time when the confidence level is high and determine a dynamic calibration curve (such as a polynomial fit). The dynamic calibration curve can associate the ratio of DC components with the wearer's calculated SpO₂. The plethysmograph module processor can utilize the dynamic calibration curve to fill in the gaps in the SpO₂ determination when the confidence levels are low (e.g., when the wearer is moving). If the wearer is moving, for example, as long as the plethysmograph module 100, 300 remains coupled to the wearer's skin surface, the trends in the change in the ratio of DC components can correlate to changes in the wearer's SpO₂. For example, when the wearer is not moving, a 5% change in the calculated ratio of DC components may correspond to a 2% change in the wearer's SpO2. When the wearer is moving and there is a 10% change in the calculated ratio of DC components, for example, the processor can use the dynamic calibration curve to predict that there is a 4% change in the wearer's SpO₂. If there is no change in the ratio of DC components in times of motion, this can represent sustained SpO₂ levels.

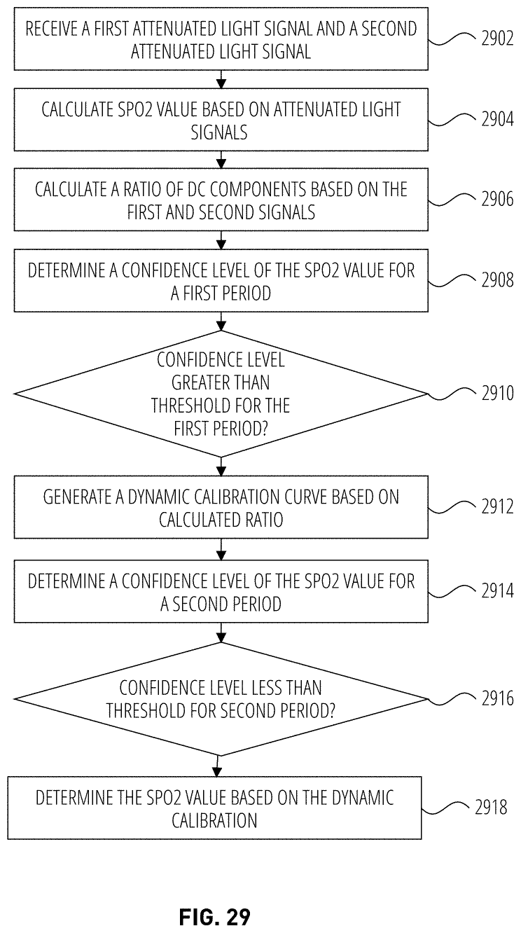

FIG. 29 illustrates an example error reduction process for SpO2 measurements that may be implemented by a controller 711. In block 2902, a controller 711 may receive a first attenuated light signal and a second attenuated light signal. In block 2904, a controller 711 may calculate SpO2 value based on attenuated light signals. In block 2906, a controller 711 may calculate a ratio of DC components based on the first and second signals. In block 2908, a controller 711 may determine a confidence level of the SpO2 value for a first period. In decision block 2910, a controller 711 may determine if a confidence level is greater than threshold for the first period. In block 2912, a controller 711 may generate a dynamic calibration curve based on calculated ratio. In block 2914, a controller 711 may determine a confidence level of the SpO2 value for a second period. In decision block 2916, a controller 711 may determine if a confidence level is less than threshold for the second period. In block 2918, determine the SpO2 value based on the dynamic calibration.

### Additional Examples

Disclosed herein are additional examples pulse recognition and blood oxygen systems and methods:

Example 1: A system for determining a plurality of physiological parameters of a user from a signal responsive to light absorption by tissue of a monitored patient, said system comprising an electronic signal processor configured to:

receive data from a plurality of sensors comprising at least one noninvasive optical sensor and at least one motion sensor, wherein said received data is responsive to light attenuated by tissue of the user; and

determine a plurality of physiological parameters using a plurality of parameter engines configured to calculate one or more physiological parameters based on the received data.

Example 2: The system of Example 1, wherein the received data comprises:

one or more optical signals comprising a red PPG signal, an IR PPG signal, and a green PPG signal; and

one or more motion signals comprising at least one gyroscope signal and at least one accelerometer signal.

Example 3: The system of Example 2, wherein the one or more optical signals comprise a red signal, an infrared signal, and a green signal.

Example 4: The system of Example 3, wherein the red signal comprises a signal with a wavelength of approximately 625 nm.

Example 5: The system of Example 3, wherein the red signal comprises a signal with a wavelength of approximately 660 nm.

Example 6: The system of Example 3, wherein the infrared signal comprises a signal with a wavelength of approximately 905 nm.

Example 7: The system of Example 3, wherein the green signal comprises a signal with a wavelength of approximately 525 nm.

Example 8: The system of Example 3, wherein the one or more optical signals further comprise a signal with a wavelength of approximately 970 nm.

Example 9: The system of Example 2, wherein the electronic signal processor is configured to normalize the one or more optical signals.

Example 10: The system of Example 9, wherein normalizing the one or more optical signals comprises taking a log of the one or more optical signals.

Example 11: The system of Example 2, wherein the electronic signal processor is configured to process the one or more motion signals to generate one or more interpolated motion signals.

Example 12: The system of Example 11, wherein to generate the one or more interpolated motion signals, the electronic signal processor is configured to:

determine time stamps associated with the one or more motion signals;

determine time stamps associated with the one or more optical signals;

interpolate motion data to align with at least some of the time stamps associated with the one or more optical signals; and

decimate the interpolated motion data to generate the one or more interpolated signals at a reduced sample rate from the interpolated motion data.

Example 13: The system of Example 12 wherein the interpolated motion data comprises at least one interpolated gyroscope signal and at least one interpolated accelerometer signal.

Example 14: The system of Example 1, wherein the plurality of physiological parameters comprises at least one of a pulse rate, SpO2 value, perfusion index, pleth variability index, respiration rate, and hydration value.

Example 15: The system of Example 14, wherein to determine a pulse rate, the electronic signal processor is configured to:

receive data comprising the one or more optical signals, the one or more normalized optical signals, and the one or more interpolated motion signals;

analyze the received data using a plurality of pulse rate engines to determine a plurality of candidate pulse rates;

analyze the plurality of candidate pulse rates using decision logic to select a candidate pulse rate to output; and

output at least one of a pulse rate, saturation information, perfusion index information, pleth variability index information, and motion information.

Example 16: The system of Example 15, wherein the plurality of pulse rate engines comprise a time domain pulse rate engine, a frequency domain pulse rate engine, and noise reduction pulse rate engine.

Example 17: The system of Example 16, wherein to analyze the received data using a noise reduction engine, the electronic signal processor is configured to, for each sub-engine:

select a first candidate pulse rate based on a first confidence score,

select a second candidate pulse rate based on a second confidence score;

select a noise reduction candidate pulse rate from the first and second candidate pulse rates; and

output the selected noise reduction candidate pulse rate as a candidate pulse rate associated with the noise reduction pulse rate engine.

Example 18: The system of Example 17, wherein the first candidate pulse rate is associated with a candidate peak in a sampled portion of a PPG spectrum, and wherein the first confidence score is based on a sum of harmonics associated with the candidate peak divided by a total energy in the sampled portion of the PPG spectrum.

Example 19: The system of Example 17, wherein the second candidate pulse rate is associated with a candidate peak in a sampled portion of a PPG spectrum, and wherein the second confidence score is based on a sum of harmonics associated with the candidate peak divided by a total energy of peaks above the candidate peak in the sampled portion of the PPG spectrum.

Example 20: The system of Example 17, wherein to select a noise reduction candidate pulse rate, the electronic signal processor is configured to:

select a first candidate pulse rate if the first candidate pulse rate passes a first threshold confidence; and

select the first candidate pulse rate if the first candidate pulse rate matches a second candidate pulse rate match within an acceptable tolerance.

Example 21: The system of Example 17, wherein to select a noise reduction candidate pulse rate, the electronic signal processor is configured to:

select a first candidate pulse rate associated with a first sub-engine if the first candidate pulse rate associated with the first sub-engine passes a first confidence threshold;

select the greater of the first candidate pulse rate associated with the first sub-engine and a first candidate pulse rate associated with a second sub-engine if:

the first candidate pulse rate from the first sub-engine and a second candidate pulse rate from the first sub-engine match,

a first and second candidate pulse rate from the second sub-engine match, and

the first candidate pulse rate from the first sub-engine and the first candidate pulse rate from the second sub-engine are within an acceptable range of each other;

select the second candidate pulse rate associated with the first sub-engine if the second candidate pulse rate passes a second confidence threshold; and

select a default value if no satisfactory pulse is otherwise selected.

Example 22: The system of Example 21, wherein the first confidence threshold is a function of pulse rate.

Example 23: The system of Example 21, wherein the acceptable range is a function of pulse rate.

Example 24: The system of Example 21, wherein the second confidence threshold is a function of pulse rate.

Example 25: The system of Example 21, wherein a default value comprises a pulse rate calculated based on a running average of recent pulse rate values.

Example 26: The system of Example 21, wherein the default value is zero.

Example 27: The system of Example 16, wherein to analyze the received data using a noise reduction engine, the electronic signal processor is configured to:

determine at least one candidate pulse rate from PPG signals in the received data using at least one of a gyroscope and accelerometer signal as a noise reference;

if a minimum peak of at least one gyroscope signal or accelerometer signal is greater than a first minimum frequency:

apply a first bandpass filter to the PPG signals to select signal above the first minimum frequency; and

determine at least one candidate pulse rate from the first filtered PPG signals in the received data using at least one of a gyroscope and accelerometer signal as a noise reference;

if a minimum peak of at least one gyroscope signal or accelerometer signal is greater than a second minimum frequency:

apply a second bandpass filter to the PPG signals to select signal above the second minimum frequency; and

determine at least one candidate pulse rate from the second filtered PPG signals in the received data using a green signal of the PPG signals as a self-reference;

determine at least one candidate pulse rate from the PPG signals using a red signal of the PPG signals as a noise reference; and

determine at least one candidate pulse rate from the PPG signals using an infrared signal of the PPG signals as a noise reference.

Example 28: The system of Example 27, wherein the first minimum frequency is based on a relationship between the accelerometer and gyroscope signals.

Example 29: The system of Example 28, wherein the minimum frequency is the lesser of a peak frequency in the gyroscope signal and the accelerometer signal where the peak frequency in the gyroscope signal and the peak frequency in the accelerometer signal have a 2 to 1 relationship.

Example 30: The system of Example 28, wherein the minimum frequency is the lesser of a peak frequency in the gyroscope signal and the accelerometer signal where the peak frequency in the gyroscope signal and the peak frequency in the accelerometer signal have a 1 to 2 relationship.

Example 31: The system of Example 27, wherein the first minimum frequency is 80 beats per minute.

Example 32: The system of Example 27, wherein the second minimum frequency is based on a relationship between the accelerometer and gyroscope signals.

Example 33: The system of Example 27, wherein the second minimum frequency is 24 beats per minute.

Example 34: The system of Example 15, wherein to analyze the plurality of candidate pulse rates using decision logic, the electronic signal processor is configured to:

select a pulse rate associated with a low motion engine if motion falls below a threshold level;

perform one or more time domain feature checks to determine the accuracy of the pulse rate;

determine a type and elapsed time of a motion present in the received data;

determine a set of boundaries for an expected pulse rate based on the type and elapsed type of the motion; and

select a pulse rate associated with a motion engine if the pulse rate falls within the set of boundaries.

Example 35: The system of Example 34, wherein the low motion engine comprises a time domain pulse rate engine or frequency domain pulse rate engine.

Example 36: The system of Example 34, wherein the motion engine comprises a noise reduction engine.

Example 37: The system of Example 14, wherein to determine an SpO2 value, the electronic signal processor is configured to:

receive data comprising one or more normalized optical signals, the pulse rate and motion information associated with one or more motion signals;

analyze the received data using a plurality of saturation engines to determine a plurality of candidate SpO2 values;

analyze the plurality of candidate SpO2 values using decision logic to select a candidate SpO2 value to output; and

output the SpO2 value.

Example 38: The system of Example 37, wherein the plurality of saturation engines comprise a seed saturation engine, an SST saturation engine, a DST saturation engine, an interference mitigation engine, and a signal noise reference saturation engine.

Example 39: The system of Example 39, wherein to analyze the received data using an interference mitigation saturation engine, the electronic signal processor is configured to:

select, based on a selected pulse rate, a correct peak in a PPG spectrum in order to generate a ratio of a red and infrared signals to determine a candidate SpO2 value.

Example 40: The system of Example 39, wherein to analyze the received data using a signal noise reference saturation engine, the electronic signal processor is configured to: use a green PPG signal as a signal reference to determine a candidate SpO2 value.

Example 41: The system of Example 39, wherein to analyze the received data using a signal noise reference saturation engine, the electronic signal processor is configured to:

project red PPG or infrared PPG signals onto green PPG signal subspace; and

subtract the projection from red PPG or infrared PPG signal to determine at least one noise reference;

use a noise canceller to remove the at least one noise reference from the red PPG signal and infrared PPG signal to generate a clean red PPG signal and clean infrared PPG signal; and

determine a candidate SpO2 value based on a ratio of the clean infrared PPG signal and clean red PPG signal.

Example 42: The system of Example 39, wherein to analyze the received data using a signal noise reference saturation engine, the electronic signal processor is configured to:

project red PPG or infrared PPG signals onto green PPG signal subspace; and

subtract the projection from red PPG signal and infrared PPG signal to determine at least one noise reference;

use a noise canceller to remove green PPG signal from the red PPG signal and infrared PPG signal to determine at least one noise reference;

use a noise canceller to remove the at least one noise reference from the red PPG signal and infrared PPG signal to generate a clean red PPG signal and clean infrared PPG signal; and

determine a candidate SpO2 value based on a ratio of the clean infrared PPG signal and clean red PPG signal.

Example 43: The system of Example 37, wherein to analyze the plurality of candidate SpO2 values using decision logic, the electronic signal processor is configured to:

determine a first candidate SpO2 value;

determine a baseline saturation tracker based on a DC component of a red PPG signal and a DC component of an infrared PPG signal;

determine if there is excessive motion interference;

output the first candidate SpO2 value if there is no excessive motion interference;

select a second candidate SpO2 value based on one or more features in the received data and the baseline saturation tracker to output; and

update the baseline saturation tracker.

Example 44: The system of Example 43, wherein to determine if these is excessive motion interference, the electronic signal processor is configured to:

determine if there is greater than a threshold amount of variation in a saturation trend based on the baseline saturation tracker.

Example 45: The system of Example 43, wherein to determine if these is excessive motion interference, the electronic signal processor is configured to:

determine if there is poor correlation across at least one of a pair of signals, the pair of channels comprising a red PPG signal and a green PPG signal, the red PPG signal and an infrared PPG signal, and the infrared PPG signal and the green PPG signal.

Example 46: The system of Example 43, wherein to determine if these is excessive motion interference, the electronic signal processor is configured to:

determine if there is motion greater than a threshold level based on one or more motion features in a gyroscope or accelerometer signal.

Example 47: The system of Example 43, wherein to determine if these is excessive motion interference, the electronic signal processor is configured to:

determine if a rate of motion is within a threshold range of a pulse rate.

Example 48: The system of Example 14, wherein to determine a hydration value, the electronic signal processor is configured to:

receive data comprising one or more normalized optical signals comprising the IR signal at 905 nm and an IR signal at 970 nm;

determine the hydration value based on the IR signal at 905 nm and the IR signal at 970 nm.

Example 49: A system for determining pulse rate of a user from a signal responsive to light absorption by tissue of a monitored patient, said system comprising an electronic signal processor configured to:

receive one or more optical signals comprising a red PPG signal, an IR PPG signal, and a green PPG signal;

receive one or more motion signals comprising at least one gyroscope signal and at least one accelerometer signal;

determine at least one first candidate pulse rate from the green PPG signal using the one or more motion signals;

if a minimum peak of the one or more motion signals is greater than a first minimum frequency:

apply a first bandpass filter to the green PPG signal to select signal above the first minimum frequency; and

determine at least one second candidate pulse rate from the first filtered green PPG signals using the one or more motion signals as a noise reference;

if a minimum peak of the one or more motion signals is greater than a second minimum frequency:

apply a second bandpass filter to the green PPG signal to select signal above the second minimum frequency; and

determine at least one third candidate pulse rate from the second filtered green PPG signal using the green PPG signal as a self-reference;

determine at least one fourth candidate pulse rate from the green PPG signal using a red signal as a noise reference;

determine at least one fifth candidate pulse rate from the green PPG signal using an infrared signal as a noise reference and select a final pulse rate from among the at least one first, second, third, fourth, and fifth candidate pulse rates.

Example 50: A system for determining pulse rate of a user from a signal responsive to light absorption by tissue of a monitored patient, said system comprising an electronic signal processor configured to:

receive one or more optical signals comprising a red PPG signal, an IR PPG signal, and a green PPG signal;

receive one or more motion signals comprising at least one gyroscope signal and at least one accelerometer signal;

determine a plurality of pulse rate candidates using a plurality of pulse rate engines;

select a first candidate pulse rate associated with a low motion engine if motion falls below a threshold level;

perform one or more time domain feature checks to determine the accuracy of the first candidate pulse rate;

determine a type and elapsed time of a motion present in the one or more optical signals or one or more motion signals;

determine a set of boundaries for a pulse rate based on the type and elapsed type of the motion;

select a second candidate pulse rate associated with a motion engine if the pulse rate falls within the set of boundaries; and

output the selected first or second candidate pulse rates as a final pulse rate.

Example 51: A system for determining an SpO2 value of a user from a signal responsive to light absorption by tissue of a monitored patient, said system comprising an electronic signal processor configured to:

receive one or more optical signals comprising a red PPG signal, an IR PPG signal, and a green PPG signal;

receive one or more motion signals comprising at least one gyroscope signal and at least one accelerometer signal;

determine a plurality of SpO2 candidate values using a plurality of saturation engines;

select a first candidate SpO2 value from the plurality of SpO2 candidate values;

determine a baseline saturation tracker based on a DC component of a red PPG signal and a DC component of an infrared PPG signal;

determine if there is excessive motion interference;

output the first candidate SpO2 value if there is no excessive motion interference;

select a second candidate SpO2 value based on one or more features in the received data and the baseline saturation tracker to output;

update the baseline saturation tracker; and

output the selected first or second candidate SpO2 values as a final SpO2 value.

Example 52: A system for determining an SpO2 value of a user from a signal responsive to light absorption by tissue of a monitored patient, said system comprising an electronic signal processor configured to:

receive one or more optical signals comprising a red PPG signal, an IR PPG signal, and a green PPG signal;

receive one or more motion signals comprising at least one gyroscope signal and at least one accelerometer signal;

project red PPG and infrared PPG signals respectively onto green PPG signal subspace; and

subtract the projection from the red PPG signal and the infrared PPG signal to determine at least one noise reference;

use a noise canceller to remove green PPG signal from the red PPG signal and infrared PPG signal to determine at least one noise reference;

use a noise canceller to remove the at least one noise reference from the red PPG signal and infrared PPG signal to generate a clean red PPG signal and clean infrared PPG signal; and

determine a candidate SpO2 value based on a ratio of the clean infrared PPG signal and clean red PPG signal.

Example 53: A system for determining an SpO2 value of a user from a signal responsive to light absorption by tissue of a monitored patient, said system comprising an electronic signal processor configured to:

receive one or more optical signals comprising a red PPG signal, an IR PPG signal, and a green PPG signal;

receive one or more motion signals comprising at least one gyroscope signal and at least one accelerometer signal;

project red PPG or infrared PPG signals onto green PPG signal subspace; and

subtract the projection from the red PPG or infrared PPG signal to determine at least one noise reference;

use a noise canceller to remove the at least one noise reference from the red PPG signal and infrared PPG signal to generate a clean red PPG signal and clean infrared PPG signal; and

determine a candidate SpO2 value based on a ratio of the clean infrared PPG signal and clean red PPG signal.

Any of the above examples may be combined.

### Terminology

Many other variations than those described herein can be apparent from this disclosure. For example, depending on the embodiment, certain acts, events, or functions of any of the algorithms described herein can be performed in a different sequence, can be added, merged, or left out altogether (for example, not all described acts or events can be necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Not necessarily all such advantages are achieved in accordance with any particular embodiment of the embodiments disclosed herein. Thus, the embodiments disclosed herein can be embodied or carried out in a manner that achieves or optimizes one advantage or group of advantages as taught herein without necessarily achieving other advantages as may be taught or suggested herein.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality can be implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a hardware processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A hardware processor can be a microprocessor, but in the alternative, the processor can be a controller, microcontroller, or state machine, combinations of the same, or the like. A processor can include electrical circuitry or digital logic circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language used herein, such as, among others, “can,” “might,” “may,” “for example,” and the like, unless specifically stated otherwise, or otherwise understood within the context as used, are generally intended to convey that certain embodiments include, while other embodiments do not include, certain features, elements and/or states. Thus, such conditional language is not generally intended to imply that features, elements and/or states are in any way may be required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” mechanism one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, can be otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments may require at least one of X, at least one of Y, or at least one of Z to each be present.

Unless otherwise explicitly stated, articles such as “a” or “an” should generally be interpreted to include one or more described items. Accordingly, phrases such as “a device configured to” is intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it is understood that various omissions, substitutions, and changes in the form and details of the devices or algorithms illustrated can be made without departing from the spirit of the disclosure. As is recognized, certain embodiments of the inventions described herein can be embodied within a form that does not provide all of the features and benefits set forth herein, as some features can be used or practiced separately from others.

What is claimed is:

1. A system for determining a plurality of physiological parameters of a user from a signal responsive to light absorption by tissue of a monitored patient, said system comprising an electronic signal processor configured to:
receive data from a plurality of sensors comprising at least one noninvasive optical sensor and at least one motion sensor, wherein said received data is responsive to light attenuated by tissue of the user; and
determine a pulse rate associated with the user using a plurality of parameter engines configured to calculate the pulse rate based on the received data.

2. The system of claim 1, wherein the received data comprises:
one or more optical signals comprising at least one red PPG signal, at least one IR PPG signal, and at least one green PPG signal; and
one or more motion signals comprising at least one gyroscope signal and at least one accelerometer signal.

3. The system of claim 1, wherein to determine a pulse rate, the electronic signal processor is configured to:
receive data comprising the one or more optical signals, the one or more normalized optical signals, and the one or more interpolated motion signals;
analyze the received data using a plurality of pulse rate engines to determine a plurality of candidate pulse rates;
analyze the plurality of candidate pulse rates using decision logic to select a candidate pulse rate to output; and
output at least one of a pulse rate, saturation information, perfusion index information, pleth variability index information, and motion information.

4. The system of claim 3, wherein the plurality of pulse rate engines comprise a time domain pulse rate engine, a frequency domain pulse rate engine, and noise reduction pulse rate engine.

5. The system of claim 4, wherein to analyze the received data using a noise reduction engine, the electronic signal processor is configured to, for each sub-engine:
select a first candidate pulse rate based on a first confidence score,
select a second candidate pulse rate based on a second confidence score;
select a noise reduction candidate pulse rate from the first and second candidate pulse rates; and
output the selected noise reduction candidate pulse rate as a candidate pulse rate associated with the noise reduction pulse rate engine.

6. The system of claim 5, wherein the first candidate pulse rate is associated with a candidate peak in a sampled portion of a PPG spectrum, and wherein the first confidence score is based on a sum of harmonics associated with the candidate peak divided by a total energy in the sampled portion of the PPG spectrum.

7. The system of claim 5, wherein the second candidate pulse rate is associated with a candidate peak in a sampled portion of a PPG spectrum, and wherein the second confidence score is based on a sum of harmonics associated with the candidate peak divided by a total energy of peaks above the candidate peak in the sampled portion of the PPG spectrum.

8. The system of claim 5, wherein to select a noise reduction candidate pulse rate, the electronic signal processor is configured to:
select a first candidate pulse rate if the first candidate pulse rate passes a first threshold confidence; and
select the first candidate pulse rate if the first candidate pulse rate matches a second candidate pulse rate match within an acceptable tolerance.

9. The system of claim 5, wherein to select a noise reduction candidate pulse rate, the electronic signal processor is configured to:
select a first candidate pulse rate associated with a first sub-engine if the first candidate pulse rate associated with the first sub-engine passes a first confidence threshold;
select the greater of the first candidate pulse rate associated with the first sub-engine and a first candidate pulse rate associated with a second sub-engine if:
the first candidate pulse rate from the first sub-engine and a second candidate pulse rate from the first sub-engine match,
a first and second candidate pulse rate from the second sub-engine match, and
the first candidate pulse rate from the first sub-engine and the first candidate pulse rate from the second sub-engine are within an acceptable range of each other;
select the second candidate pulse rate associated with the first sub-engine if the second candidate pulse rate passes a second confidence threshold; and
select a default value if no satisfactory pulse is otherwise selected.

10. The system of claim 9, wherein the first or second confidence threshold is a function of pulse rate.

11. The system of claim 9, wherein a default value comprises a pulse rate calculated based on a running average of recent pulse rate values.

12. The system of claim 9, wherein the default value is zero.

13. The system of claim 4, wherein to analyze the received data using a noise reduction engine, the electronic signal processor is configured to:
determine at least one candidate pulse rate from PPG signals in the received data using at least one of a gyroscope and accelerometer signal as a noise reference;
if a minimum peak of at least one gyroscope signal or accelerometer signal is greater than a first minimum frequency:
apply a first bandpass filter to the PPG signals to select signal above the first minimum frequency; and
determine at least one candidate pulse rate from the first filtered PPG signals in the received data using at least one of a gyroscope and accelerometer signal as a noise reference;
if a minimum peak of at least one gyroscope signal or accelerometer signal is greater than a second minimum frequency:
apply a second bandpass filter to the PPG signals to select signal above the second minimum frequency; and
determine at least one candidate pulse rate from the second filtered PPG signals in the received data using a green signal of the PPG signals as a self-reference;
determine at least one candidate pulse rate from the PPG signals using a red signal of the PPG signals as a noise reference; and
determine at least one candidate pulse rate from the PPG signals using an infrared signal of the PPG signals as a noise reference.

14. The system of claim 13, wherein the first minimum frequency is based on a relationship between the accelerometer and gyroscope signals.

15. The system of claim 14, wherein the minimum frequency is the lesser of a peak frequency in the gyroscope signal and the accelerometer signal where the peak frequency in the gyroscope signal and the peak frequency in the accelerometer signal have a 2 to 1 relationship.

16. The system of claim 14, wherein the minimum frequency is the lesser of a peak frequency in the gyroscope signal and the accelerometer signal where the peak frequency in the gyroscope signal and the peak frequency in the accelerometer signal have a 1 to 2 relationship.

17. The system of claim 13, wherein the second minimum frequency is based on a relationship between the accelerometer and gyroscope signals.

18. The system of claim 3, wherein to analyze the plurality of candidate pulse rates using decision logic, the electronic signal processor is configured to:
select a pulse rate associated with a low motion engine if motion falls below a threshold level;
perform one or more time domain feature checks to determine the accuracy of the pulse rate;
determine a type and elapsed time of a motion present in the received data;
determine a set of boundaries for an expected pulse rate based on the type and elapsed type of the motion; and
select a pulse rate associated with a motion engine if the pulse rate falls within the set of boundaries.

19. The system of claim 18, wherein the low motion engine comprises a time domain pulse rate engine or frequency domain pulse rate engine.

20. The system of claim 18, wherein the motion engine comprises a noise reduction engine.
